(19) United States
(12) Patent Application Publication
(10) Pub. No.: US 2024/0228952 A9
(43) Pub. Date: Jul. 11, 2024

(54) APPARATUS AND METHOD FOR COLLECTION OF SPERM SAMPLES

(71) Applicants: Texas Tech University System, Lubbock, TX (US); RSI Technology Group, LLC, Dallas, TX (US)

(72) Inventors: Lindsay L. PENROSE, Lubbock, TX (US); Samuel D. PRIEN, Shallowater, TX (US)

(73) Assignees: TEXAS TECH UNIVERSITY SYSTEM, Lubbock, TX (US); RSI TECHNOLOGY GROUP, LLC, Dallas, TX (US)

(21) Appl. No.: 18/482,725

(22) Filed: Oct. 6, 2023

Related U.S. Application Data
(60) Provisional application No. 63/418,368, filed on Oct. 21, 2022.

(51) Int. Cl. C12N 5/076; A01N 1/02

(52) U.S. Cl. CPC C12N 5/061; A01N 1/0226; A01N 1/0231; A01N 1/0263

(57) ABSTRACT

The present invention relates to a method and apparatus for passive collection of sperm having improved motility from a semen sample. The semen sample is contacted with on side of a porous barrier and a nutrient-containing media is contacted with the other side of the porous barrier. More robust sperm migrate from the semen sample through the porous barrier and are collected in a nutrient-containing media.

### CROSS-REFERENCE TO RELATED APPLICATIONS

This application claims priority of U.S. Provisional Patent Application Ser. No. 63/418,368, entitled “APPARATUS AND METHOD FOR COLLECTION OF SPERM SAMPLES,” filed on Oct. 21, 2022, the contents of which are incorporated by reference herein in their entirety.

### FIELD OF THE INVENTION

The present invention relates to methods and apparatus for collection of sperm samples having improved viability.

### BACKGROUND OF THE INVENTION

Assisted reproductive technologies were developed originally to treat individuals with obstructed ovarian tubes, but have matured to procedures which, according to the U.S. Center for Disease Control (2019), now accounts for up to 2% of the annual U.S. birth rate. Since the first human birth from in vitro fertilization in 1978, there have been significant improvements in stimulation protocols, fertilization and culture techniques, use of donor gametes and embryos, and patient selection. Further, the use of pre-implantation genetic diagnosis/pre-implantation genetic screening, an invasive harvesting of cells for genetic screening, has allowed improved selection of embryos to avoid aneuploidy and other genetic defects. These improvements resulted in constantly increasing pregnancy rates (2019 Assisted Reproductive Technology—Fertility Clinic and National Summary Report).

U.S. Pat. No. 6,864,046, incorporated by reference herein in its entirety, discloses the use of a method for collecting the semen of an animal via a semen collection vessel having a semen extender solution capable of extending motility of collected semen.

U.S. Pat. No. 9,157,063, incorporated by reference herein in its entirety, discloses the use of 40 μm-to 70 μm strainer baskets and different pH sub-environments for separating sperm bearing X or Y chromosomes in collected semen, and centrifuges the sperm sample prior to initiating the separation.

U.S. Pat. No. 11,399,811, incorporated by reference herein in its entirety, discloses an apparatus comprising a combination of antioxidants and method of using same for extending the viability of collected semen.

U.S. Pat. Pub. 2021/0339253 discloses a multi-step process to isolate target cells in a biological sample using a microfluidic device comprising magnetic beads having a recognition reagent on their outer surface for binding target cells.

In addition to these traditional motivations, the recent pandemic, among other reasons, has resulted in behavioral changes promoting a need for improved preservation of semen samples. It has been found that a shift for collection at clinic to at home for the population examined resulted in an increase in the time from collection to completed preparation. This has been shown to significantly affect the viability of useful sperm samples. In addition, once a semen sample has been acquired, the complexity and equipment required to isolate motile sperm for use in general analysis, invitro-fertilization procedures, and the like is challenging and inefficient. In fact, in many of the options a number of viable motile sperm are rendered useless by the very methods used to isolate them. Therefore, there is a need for methods and apparatus for sperm collection that improve the viability and motility of sperm to be used for fertilization or testing.

Although there have been many advances in assisted reproductive technologies, there is still a need for improved devices and methods for collecting sperm samples having a higher concentration of motile sperm. Ideally, such devices would be easy to use, and such methods could be implemented with simple equipment and non-invasive techniques.

### SUMMARY OF THE INVENTION

In general, the present disclosure relates to apparatuses and methods for collection of sperm samples having improved motility or for increasing the quantity and quality of motile sperm for use in procedures such as testing and/or fertilization procedures. In particular, apparatus and methods for collection of mammalian semen samples are disclosed herein.

Disclosed herein is an apparatus for passively isolating a population of motile sperm from a solution comprising a semen sample. The apparatus comprises a first vessel for receiving the solution, and a sperm isolation device (“SID”) for receiving a portion of a media, preferably a fresh media. The SID comprises a frame and a porous barrier that form a chamber having an opening at the upper end to facilitate addition of a fluid and/or withdrawal of fluid from the chamber. The porous barrier comprises a plurality of holes, each having a passthrough dimension or a hydraulic diameter in the range of from 6 microns to 30 microns.

In some embodiments, the first vessel, or sample container, has a reservoir suitable for receiving the solution, and the SID has a chamber suitable for receiving the portion of the media. The SID fits within the sample container such that the reservoir and the chamber are fluidly connected through at least a portion of the holes in the porous barrier when the reservoir has received solution comprising a semen sample, the chamber has received a portion of media, and the SID is engaged with the sample container. In some embodiments, the SID is engaged with the sample container when the SID is self-supporting or otherwise supported by the inner surface of the sample container to keep the SID stationary in an upright orientation. Further, the SID is positioned relative to the first vessel such that after the reservoir in the first vessel has received a desired amount of solution comprising a semen sample and the chamber in the SID has received a desired amount of nutrient media, the porous barrier or at least a portion of the porous barrier is contacting the solution on one side and the media on the other side and the solution and the media may be in contact through the holes in the porous barrier. In some embodiments of the apparatus, the SID is configured to promote contact of the fresh media in the chamber of the SID and semen sample solution in the reservoir of the sample container through the porous barrier.

In another aspect, a method for passively isolating a population of motile sperm from a solution comprising a semen sample (or semen sample solution) is disclosed herein. The method comprises introducing the semen sample into a first vessel, or sample container, wherein the semen sample has a first population of sperm having a range of motility. The method further comprises introducing a nutrient media, preferably a fresh media, into a SID comprising a frame and a porous barrier, wherein the frame and porous barrier form a chamber having an opening at its upper end to facilitate addition and removal of fluids. The method further comprises lowering the SID into the sample container such that the porous barrier contacts the sample solution, is partially submerged into the sample solution, or is fully submerged in the sample solution to submerge, or partially, substantially or totally lower, at least a portion of the porous barrier. In some embodiments, the rate of lowering and/or the depth of lowering are controlled to prevent or limit breakthrough of the first media into the sample solution and/or the sample solution into the chamber. The porous barrier comprises a plurality of holes having a passthrough dimension or a hydraulic diameter in the range of from 6 microns to 30 microns. A population of motile sperm in the semen sample migrate by their own motion through the holes in the porous barrier into the first media to form a second solution comprising the media and a second population of motile sperm. After a threshold time period, a portion of the second solution is withdrawn from the SID, wherein the second solution comprises a concentration of motile sperm.

In another aspect, a sperm isolation device (“SID”) for passively isolating a population of motile sperm from a solution comprising a semen sample is disclosed. The SID comprises a frame and a porous barrier to form a chamber suitable for containing a liquid, wherein the porous barrier comprises a plurality of holes having a passthrough dimension or a hydraulic diameter in the range of from 6 microns to 30 microns.

In another aspect, a SID and method for using the SID for passively isolating a population of motile sperm from a solution comprising a semen sample is disclosed. In some embodiments, the SID is self-supporting and configured to be used with any container that will allow insertion of the SID and can hold a solution containing a semen sample at a predetermined level.

In another aspect, the invention is directed to a method for collection of motile sperm, the method comprising: a) providing a semen sample comprising a first population of sperm having a first average motility, optionally in a solution with a first media such as, but not limited to, an appropriate cell culture media or a buffered media; b) introducing a sufficient amount of a second media into the chamber of an SID such that the second media contacts the chamber side of the porous barrier, wherein the second media is fresh media such as, but not limited to, an appropriate cell culture media or a buffered media, and the second media can be the same as or different from the first media; c) lowering the SID into the semen sample sufficiently to contact the opposite side of the porous barrier with the semen sample; and d) maintaining the SID in a stationary position for a threshold time to allow motile sperm to migrate from the semen sample to the second media by their own motion to produce a second solution comprising the second media and a second motile sperm, wherein the second population of sperm has a higher average motility than the first population of sperm. In some embodiments, the porous barrier comprises holes of between 6 microns and 30 microns that extend from one surface of the porous barrier to the opposite surface of the porous barrier. In some embodiments, the method comprises the same steps wherein step b) and step c) are reversed in order. In some embodiments, the method further comprises centrifuging a portion of the second solution and recovering a third population of sperm.

In some embodiments, a method for collecting motile sperm from a semen sample for use in in-vitro fertilization comprises the steps of: a) obtaining the semen sample having a first sperm population; b) contacting one side of a porous barrier comprising a plurality of holes with the semen sample, wherein the holes have a passthrough dimension or a hydraulic diameter in the range of from 6 microns to 30 microns; c) contacting the other side of the porous barrier with a nutrient media; d) maintaining such contact and keeping the semen sample, the media, and the porous barrier stationary for a threshold period; and e) recovering a second population of sperm from the nutrient media on the opposite side of the porous barrier from the semen sample, wherein the second population of sperm has a higher average motility than the first population of sperm.

In some embodiments, a method for collection of sperm having improved motility comprises: a) providing an apparatus comprising a first vessel having a collection funnel at the upper end of the first vessel, or sample container, and a reservoir at the lower end of the sample container; b) adding a first sample comprising sperm having a first motile sperm population to the reservoir; c) engaging a SID comprising a support member with the collection funnel, wherein a SID is slidably connected to the support member, and the SID has a cylindrical frame and a porous barrier at its lower end to form a chamber; d) contacting the lower surface of the porous barrier with the first sample in the reservoir by sliding the SID within the SID support; e) adding a nutrient-containing media to the chamber of the SID in an amount sufficient to cover an upper surface of the porous barrier; and f) producing a second sample comprising the nutrient media on the upper surface of the porous barrier and at least a portion of the first motile sperm population. In some embodiments, the method comprises the same steps, except that step e) is performed before step c) —i.e., the nutrient media is added to the chamber of the SID prior to contacting the porous barrier with the semen sample. In some embodiments, the SID comprises a frame and porous barrier to form a chamber having a cylindrical, conical, frustoconical, or similar shape, wherein the porous barrier is on the side of the chamber and has a plurality of holes having a passthrough dimension or a hydraulic diameter in the range of from 6 microns to 30 microns. In such embodiments, the SID and a separate or integral support is configured to engage with the collection funnel such that the porous barrier is submerged in the semen sample when the semen sample is added to the reservoir to a predetermined level.

In some embodiments, an apparatus comprises: a) a first vessel, or sample container, having a reservoir at the lower end of the sample container; b) a SID having a cylindrical frame and a porous barrier at its lower end to form a chamber, wherein the porous barrier has a plurality of holes having a passthrough dimension or a hydraulic diameter in the range of from 6 microns to 30 microns; and c) a SID support, configured to position the bottom of the SID within the reservoir.

In some embodiments, an apparatus comprises: a) a first vessel, or sample container, having a reservoir at the lower end of the sample container; b) a SID comprising a frame and porous barrier to form a chamber having a cylindrical, conical, frustoconical, or similar shape, wherein the porous barrier is on the side of the chamber and has a plurality of holes having a passthrough dimension or a hydraulic diameter in the range of from 6 microns to 30 microns; and c) a SID support, configured to position the bottom of the SID within the reservoir of the sample container.

In some embodiments, an apparatus for enhancing the population of motile sperm from a semen sample comprises a first vessel for receiving the semen sample and a SID comprising a frame and porous barrier forming a chamber, wherein the SID is configured to receive nutrient media in the chamber. The porous barrier comprises a plurality of holes having passthrough dimension or a hydraulic diameter between 6 microns to 30 microns. The SID fits within the first vessel, or sample container, such that the porous barrier is position to contact the semen sample on one side and the nutrient media on the other side. In operation, the porous barrier is maintained in a stationary position to permit passive forward progression of motile sperm from the semen sample side of the porous barrier into the nutrient media in the chamber of the SID.

In some embodiments, the invention is directed to a method for enhancing the population of motile sperm from a semen sample, the method comprising the steps of: (i) introducing the semen sample into a first vessel, or sample container; (ii) introducing nutrient media into the chamber of the SID; and (iii) lowering the SID into said semen sample, wherein the porous barrier has a plurality of holes having a passthrough dimension or a hydraulic diameter in the range of from 6 microns and 30 microns, and the SID and sample container are configured to position the porous barrier to contact the semen sample on one side and the nutrient sample on the other side. When so positioned, motile sperm in the semen sample passively and forwardly progress through the holes in the porous barrier into the nutrient media in the chamber of the SID.

In some embodiments, the invention is directed to a SID, comprising a frame and porous barrier to form a chamber, for use in enhancing the population of motile sperm from a semen sample by allowing for the passive forward progression of motile sperm through the porous barrier into nutrient media in the chamber of the SID, wherein the porous barrier has a plurality of holes having passthrough dimension or a hydraulic diameter in the range of from 6 microns to 30 microns or from 6 microns to 25 microns.

The foregoing has outlined rather broadly the features and technical advantages of the present invention in order that the detailed description of the invention that follows may be better understood. Additional features and advantages of the invention will be described hereinafter, which form the subject matter of the claims of the invention. It should be appreciated by those skilled in the art that the conception and specific embodiments disclosed may be readily utilized as a basis for modifying or designing other film structures and/or processes for carrying out the same purposes of the present invention. It should also be realized by those skilled in the art that such equivalent constructions do not depart from the spirit and scope of the invention as set forth in the appended claims. The novel features which are believed to be characteristic of the invention, both as to its structure and method of manufacture, together with further objects and advantages will be better understood from the following description.

### BRIEF DESCRIPTION OF THE DRAWINGS

The present disclosure is illustrated by way of example, and not by way of limitation, in the accompanying drawings, in which like reference characters refer to like parts throughout, and in which:

FIG. 1 shows a vertical cross-section of an embodiment employing a sperm isolation device (SID) in single-vessel apparatus according to embodiments of the invention;

FIG. 2 shows an expanded view of the components of an embodiment employing a SID in single-vessel apparatus according to embodiments of the invention;

FIG. 3 shows a perspective view of a self-supporting SID according to embodiments of the invention;

FIG. 4 shows an elevation view of a self-supporting SID according to embodiments of the invention;

FIG. 5 shows a vertical cross-section view of a self-supporting SID in a single-vessel apparatus according to embodiments of the invention;

FIG. 6 shows a vertical cross-section of an embodiment employing a SID in two-vessel apparatus according to embodiments of the invention;

FIG. 7 shows an expanded view of the components of an embodiment employing a SID in two-vessel apparatus according to embodiments of the invention;

FIG. 8 shows a perspective view of a self-supporting SID according to embodiments of the invention;

FIG. 9 shows an elevation view of a self-supporting SID according to embodiments of the invention;

FIG. 10 shows a vertical cross-section view of a self-supporting SID in a two-vessel apparatus according to embodiments of the invention;

FIG. 11 shows two-vessel apparatus and a SID separate from one another as a step according to the methods disclosed herein;

FIG. 12 shows a SID being lowered into a two-vessel apparatus as a step according to the methods disclosed herein;

FIG. 13 shows SID engaged with a two-vessel apparatus as a step according to the methods disclosed herein;

FIG. 14 shows a graph of recovered motility versus time comparing the motile sperm recovery method disclosed herein to a conventional swim-up technique;

FIG. 15 shows a graph of recovered cells versus time comparing the motile sperm recovery method disclosed herein to a conventional swim-up technique;

FIG. 16 shows a graph of rate of motile cell recovery versus time comparing the motile sperm recovery method disclosed herein to a conventional swim-up technique;

FIG. 17 shows a graph of recovered cell concentration versus time comparing the motile sperm recovery method disclosed herein to a conventional swim-up technique;

FIG. 18 shows a graph of total motile cells recovered versus time comparing the motile sperm recovery method disclosed herein to a conventional swim-up technique;

FIG. 19 shows a graph of concentration versus time comparing the motile sperm recovery method disclosed herein to a conventional swim-up technique using frozen samples;

FIG. 20 shows a graph of motility versus time comparing the motile sperm recovery method disclosed herein to a conventional swim-up technique using frozen samples; and

FIG. 21 shows a graph of total motile cells recovered versus time comparing the motile sperm recovery method disclosed herein to a conventional swim-up technique using frozen samples.

While the disclosed process and composition are susceptible to various modifications and alternative forms, the drawings illustrate specific embodiments herein described in detail by way of example. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF THE INVENTION

Illustrative embodiments of the subject matter claimed below will now be disclosed. In the interest of clarity, some features of some actual implementations may not be described in this specification. It will be appreciated that in the development of any such actual embodiments, numerous implementation-specific decisions must be made to achieve the developer's specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the disclosure.

The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than the broadest meaning understood by skilled artisans, such a special or clarifying definition will be expressly set forth in the specification in a definitional manner that provides the special or clarifying definition for the term or phrase. It must also be noted that, as used in the specification and the appended claims, the singular forms “a,” “an,” and “the” include plural references unless otherwise specified.

For example, the following discussion contains a non-exhaustive list of definitions of several specific terms used in this disclosure (other terms may be defined or clarified in a definitional manner elsewhere herein). These definitions are intended to clarify the meanings of the terms used herein. It is believed that the terms are used in a manner consistent with their ordinary meaning, but the definitions are nonetheless specified here for clarity.

### Definitions

As used herein, “3D printing” means a process of making a physical object from a three-dimensional digital model, typically by laying down many thin layers of a material in succession.

As used herein, “blow molding” means a manufacturing process that allows hollow plastic parts to be formed. Air pressure is used to inflate soft plastic into a mold cavity. The three main types of blow molding are: extrusion blow molding, injection blow molding, and injection stretch blow molding.

As used herein, “first solution” or “solution comprising a semen sample” or “semen sample solution” and variations thereof, refers to a raw semen sample and optionally a nutrient media—i.e., a semen sample prior to processing according to the method disclosed herein. In some embodiments, the solution ranges from 100% semen to 50% semen and 50% nutrient media (volume %), wherein the nutrient media is a nutrient media as described herein and is also referred to as “sample media.”

As used herein, “harvest media” means a nutrient media as described herein that is a fresh media into which motile sperm will migrate from a semen sample solution. After such migration, the harvest media will comprise motile sperm.

As used herein, “hole” means, is equivalent to, and is interchangeable with aperture, channel, perforation, piercing, pore, slot, or gap in that it creates an opening or pathway from one surface of a porous barrier to the opposing surface of the porous barrier. In some embodiments, a hole is formed during manufacture of the porous barrier material, such as, but not limited to, gaps in a woven fabric. In some embodiments, a hole is formed after manufacture of the porous barrier material, such as, but not limited to, manual perforation of a solid material.

As used herein, “hydraulic diameter (D_{H})” provides an equivalent diameter for analysis of fluid flow in a non-circular hole. D_{H}=4A/P, where A is the cross-sectional area of the hole, and P is the wetted perimeter of the cross-section. For a circular hole, hydraulic diameter and the diameter of the circle are the same. Hydraulic diameter normalizes holes having non-circular geometric shapes such as a triangle, a square, a rectangle, a hexagon, etc., or for irregular shapes. In some embodiments, wherein a non-circular hole has a maximum dimension along a first line passing through the centroid of the hole and a transverse dimension measured along a second line through the centroid of the hole and perpendicular to the first line, the ratio of the maximum dimension to the transverse dimension is less than or equal to 3.0, less than or equal to 2.5, less than or equal to 2.0, or less than or equal to 1.5.

As used herein, “injection molding, means a manufacturing process where material is fed into a heated barrel where it is also melted. When smooth enough, the material is injected through a nozzle under pressure (filling cycle) to fill a mold cavity and then cools off (cooling cycle). Thereafter, the mold opens and the part ejects.

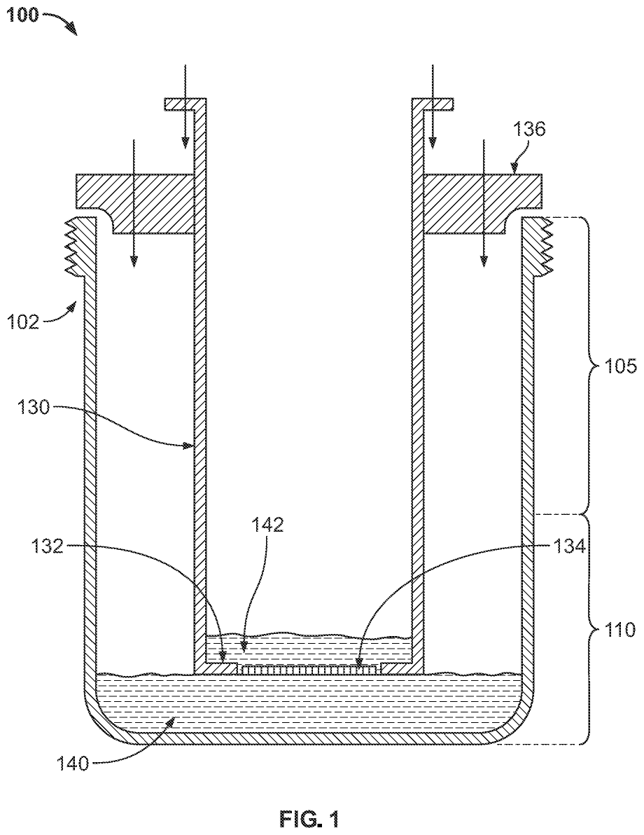

As used herein, “lower end,” with respect to the apparatus or component of the apparatus means the portion of apparatus or component of the apparatus, respectively, proximate to the bottom of the apparatus or component of the apparatus, respectively, when the apparatus is in the upright orientation resting on a horizontal surface. FIG. 1-FIG. 13 each show embodiments of the apparatus in the upright orientation.

As used herein, “migration” means forward movement of motile sperm by their own natural motion in a first fluid through a porous barrier to a second fluid on the other side of a porous barrier. The forward motion of each sperm cell is determined by the motility of such sperm cell—i.e., the sperm swim on their own through the holes in the porous barrier into the fresh media. Thus, sperm cells having a higher motility migrate farther and/or more quickly on their own volition than do sperm cells with a lower motility. In some embodiments, such migration is induced by chemotaxis, wherein motile sperm in a first fluid are attracted to move on their own volition to a second fluid, having a different chemical composition than the first fluid, on the other side of a porous barrier as discussed in more detail below. In some embodiments, motile sperm are identified as sperm having sufficiently high values of one or more of amplitude of lateral head or lateral displacement (“ALH”), average pathway velocity or path velocity (“VAP”), beat cross frequency (“BCF”), elongation, mitochondrial intactness, and morphology. Further, in the method and apparatus disclosed herein, the holes are sized to permit migration of sperm having sufficient motility from one side of the porous barrier to the other side, while impeding the migration of sperm having lower progressive motility from one side of the porous barrier to the other side.

As used herein, “passthrough dimension” means the diameter of the largest sphere that could pass through a hole in a porous barrier. The use of a sphere of a certain diameter is merely a modeling technique to describe a holes that can be straight, curved, and/or branched slots, wherein the width of the slot is the passthrough dimension.

As used herein, “porous barrier” means a selective barrier comprising a plurality of holes having a specified hydraulic diameter as discussed in more detail elsewhere herein. The term “porous barrier” is equivalent to and interchangeable with the term “interface sieve.” A porous barrier could be considered a passive and stationary interface sieve in that it is located at and assists in maintaining the interface and between the solution comprising the semen sample and the media to attract motile sperm and prevents passage of unwanted entities to the other side. The holes are sized such that when the porous barrier is positioned between a first fluid and a second fluid, the porous barrier maintains or substantially maintains the interface between the first and second fluid by minimizing or preventing either fluid from flowing to the other side of the porous barrier. Further, in the method and apparatus disclosed herein, the holes are sized to permit migration of sperm having sufficient motility from one side of the porous barrier to the other side, while impeding the migration of sperm having lower motility or no motility including sperm fragments from one side of the porous barrier to the other side. In some embodiments, such migration is induced by chemotaxis and/or other physical properties, wherein higher motility sperm in a first fluid are attracted to a second fluid on the other side of a porous barrier, wherein the second fluid has a nutrient concentration, a pH level, selected chemical compounds, or a combination thereof to attract motile sperm. The porous barrier can be formed from any material that prevents or minimizes interaction, reaction, and/or damage to sperm cells. In some embodiments, the porous barrier comprises a mesh or matrix of material, which inherently has a plurality of holes, or a sheet that is perforated either during or after manufacture to add plurality of holes. Such porous barriers are each added to a frame to form a sperm isolation device. In some embodiments, the porous barrier is formed integrally into the sperm isolation device, wherein a region of the device comprises a plurality of holes implemented either during or after manufacture of the sperm isolation device. In some embodiments, when in operation, the porous barrier, the first fluid, and the second fluid remain motionless or substantially motionless with respect to an external force, and the only motion is at the cellular level, wherein the sperm cells are self-propelled. That is to say, the porous barrier is a passive device that remains stationary during migration of motile sperm through the holes in the porous barrier.

As used herein, “second solution” or “nutrient media comprising enriched sperm” and variations thereof, refers to a solution comprising a nutrient media and motile sperm that has passed through the porous barrier of a sperm isolation device according to the methods disclosed herein.

As used herein, “sperm isolation device” or “SID” means a SID comprising a frame and a porous barrier that form a chamber suitable for containment of a fluid, such as a semen sample or a nutrient media. In some embodiments, the frame and porous barrier are separate components, wherein the frame can be any geometric configuration, provided that after assembly with the porous barrier, a chamber is formed, such that when a desired amount of fluid is added to the chamber, all or at least a portion of the porous barrier will be in contact with the fluid. In some embodiments, the frame and porous barrier are integral to form a chamber—i.e., the frame comprises one or more regions having a plurality of holes or the porous barrier is formed from a self-supporting material in the shape of a chamber, such that when a desired amount of fluid is added to the chamber, all or at least a portion of the plurality of holes in the former or the porous barrier in the latter will be in contact with the fluid. In one embodiment, the porous barrier is a SID in which the SID is made from a solid material, i.e., a plastic by any of the methods described above, and hole are formed either directly through one of the methods described above, or simply formed indirectly through the SID.

As used herein, “thermoplastic” means any polymer including but not limited to acrylonitrile butadiene styrene (“ABS”), polyamide (“PA”), polybutylene terephthalate (“PBT”), polycaprolactam, polycarbonate (“PC”), polyether ether ketone (“PEEK”), polyetherimide, polyethylene (“PE”), polyethylene terephthalate (“PETP”), polymethyl methacrylate (“PMMA”), polyoxymethylene (“POM”), polyphenylene sulfide (“PPS”), polyphenylsulfone, polypropylene (“PP”), polystyrene (“PS”), polyvinylidene fluoride (“PVDF”), styrene acrylonitrile resin (“SAN”), thermoplastic elastomers (“TPE”), thermoplastic polyurethane (“TPU”), or combinations thereof.

As used herein, “upper end” with respect to the apparatus or component of the apparatus means the portion of apparatus or component of the apparatus, respectively, proximate to the bottom of the apparatus or component of the apparatus, respectively, when the apparatus is in the upright orientation resting on a horizontal surface. FIG. 1-FIG. 13 each show embodiments of the apparatus in the upright orientation.

### Method for Collection of Sperm Samples

In some embodiments, a method for collection of motile sperm comprises providing a semen sample comprising a sperm population having an initial vitality. In some embodiments, the initial vitality can be measured as a first motile population, a first normal morphology population, or a combination thereof. Collection of the semen sample can be implemented by any convenient means that permits application of the method described herein and/or use of an apparatus as described herein. In some embodiments, a semen sample is deposited in the apparatus directly from the donor, wherein nutrient media, particularly a buffered media, is optionally introduced into the apparatus before, with, and/or after the semen sample. In some embodiments, a semen sample is collected remotely from the apparatus and subsequently transferred to the apparatus alone or in combination with a nutrient media.

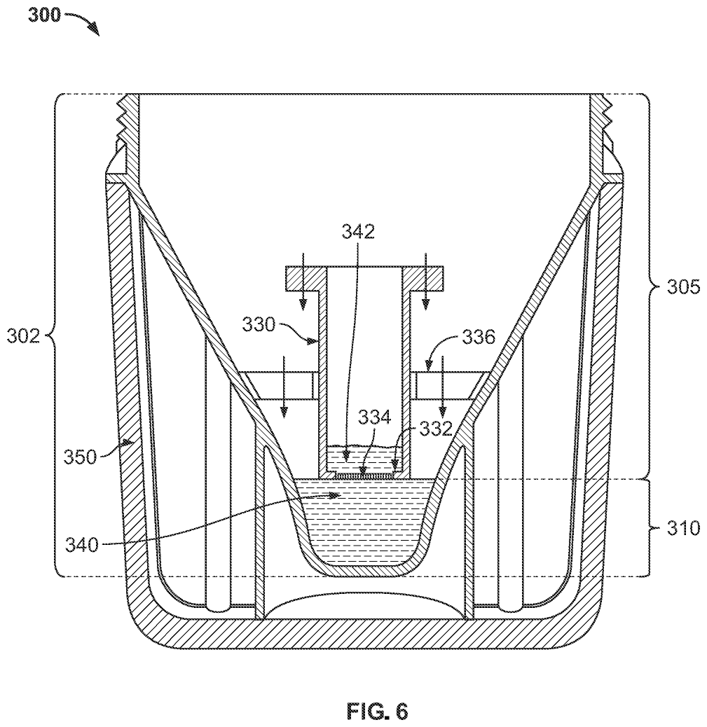

FIG. 1-FIG. 5 show a sample collection device referred to herein as a Type 1 device. FIG. 6-FIG. 10 show a sample collection device referred to herein as a Type 2 device. In some embodiments, a semen sample can be collected directly in any container that is specifically designed for collection and/or storage of sperm samples, such as, but not limited to, a Type 1 or Type 2 device. In some embodiments, a semen sample can be collected at a desired time and place and then be transported for processing in a Type 1 or Type 2 device at a different time and place.

In some embodiments, a semen sample can also be collected in any container that is not specifically designed for collection and/or storage of semen samples (“non-standard container”), provided that such container facilitates use of the method and/or apparatus as described herein. Such generic containers can include test tubes, petri dishes, cups formed from paper, extruded foamed polystyrene, thermoplastic, or any other material, provided that the inner surface of the container will cause no damage or very limited damage to the sperm sample. This can be accomplished by either forming the container from a material meeting this qualification or coating the inner surface of the container with a material meeting this qualification. In some embodiments, the semen sample further comprises a nutrient media, optionally a buffered media. In some embodiments, methods described herein can be implemented directly using a non-standard container, provided that the non-standard container is of a size and shape to permit use of a stand-alone sperm isolation device as later described herein.

A semen sample, with or without added nutrient media, as added to the reservoir of the first vessel, or sample container. A sperm isolation device (“SID”) is provided, wherein the SID comprises a frame and a porous barrier to form a chamber having any opening at its upper end suitable for adding or withdrawing fluid. The SID is lowered into the solution comprising the semen sample, either before or after a nutrient media is added to the chamber of the SID. In some embodiments, the ratio of the volume of the sperm sample solution to the volume of the nutrient media in the chamber is in the range of from 2:3 to 10:1, from 1:1 to 8:1, or from 2:1 to 6:1 for human samples. One skilled in the art would understand the ratio of the volume of the sperm sample solution to the volume of the nutrient media in the chamber to produce sufficient concentration of motile sperm in the nutrient media in the chamber of the SID for different artificial insemination processes. For example, in in vitro fertilization (IVF) requires a sperm concentration in the second solution of at least 100 k/mL, preferably 500 k/mL, in vitro fertilization with intracytoplasmic sperm injection (ICSI)) requires a sperm concentration in the second solution of at least 100 k/mL, and intrauterine insemination (IUI)) requires a sperm concentration in the second solution of at millions/mL. The concentration of sperm in the second solution is further of function of concentration of sperm in an original semen sample, which varies by species as shown in Table 1 below. For semen samples from other species, one skilled in the art would understand how to adjust these ratios to account for different sperm concentrations in animal semen samples in order to produce a sufficient concentration of motile sperm in the nutrient media in the chamber of the SID. The nutrient media added to the chamber of the SID can be the same as or different from the nutrient media added to the semen sample, if any. The SID is lowered to a point where the porous barrier contacts the surface of the semen sample, is partially submerged in the semen sample, or is fully submerged in the semen sample. The SID is configured to engage with the inner surface of the sample container through an integral or separate support member. In some embodiments, the elevation of the SID relative to the level of the semen sample in the reservoir is controlled by setting a target level for the amount of semen sample to be added to the reservoir, a vertically slidable attachment between the SID and a separate SID support member, or a combination thereof. After the SID is engaged with the inner surface of the sample container, and optionally adjusted vertically, such that the porous barrier is contacting the surface of or immersed in the semen sample, the porous barrier will have contact with the semen sample on its lower surface and contact with the nutrient media on its upper surface. The porous barrier has a plurality of holes having a passthrough dimension or hydraulic diameter in the range of from 6 microns to 30 microns that enables fluid connection between the reservoir of the sample container and the chamber of the SID. While the porous barrier produces an effective barrier to prevent mass flow of fluids between the reservoir and the chamber, motile cells can penetrate the barrier by traversing through fluid in the holes.

In some embodiments, a sperm isolation device (“SID”) comprises a frame extending downwardly to a porous barrier at the lower end of the frame, wherein the porous barrier can be flat or substantially flat. In some embodiments, the porous barrier can be concave or convex with respect to the surface of the solution comprising the semen sample. In such embodiments, the shape of the porous barrier can be the shape of a hemispherical surface, a portion of a hemispherical surface, an elliptical surface, a portion of an elliptical surface, a conical surface, a frustoconical surface, or any other geometric configuration suited to provide increased surface area contact with the sperm sample when compared to a flat porous barrier having a perimeter of the same size and shape. In operation of the methods disclosed herein, the SID is engaged by a support means to position the porous barrier at the surface or below the surface of a solution comprising a semen sample. A fresh nutrient media is added to the chamber of the SID before or after contacting the porous barrier with the solution.

In some embodiments, a sperm isolation device (“SID”) comprises a frame having a ring member attached to a solid base by at least two downwardly extending supports, wherein the porous barrier is positioned between each pair of adjacent supports and extends from the solid base to the rim member to form a chamber suitable for receiving the portion of fluid, such as fresh nutrient media. In such embodiments, the chamber can be cylindrical or frustoconical, wherein at least a portion of the sidewall of the chamber comprises the porous barrier. In such embodiments, in operation of the methods disclosed herein, the SID is partially submerged in a fluid, such as a solution comprising a semen sample. A fresh nutrient media is added to the chamber of the SID before or after contacting the porous barrier with the solution.

It is believed, without wishing to be bound by any particular theory, that surface tension of the solution and/or the fresh nutrient media prevents or substantially limits passage of the solution to the chamber side of the porous barrier, i.e., through the holes, or passage of the fresh nutrient media through holes to the solution side of the porous barrier. This is with the proviso that insertion of the SID into the solution and addition of fresh media are performed in a delicate manner so as to preserve the surface tension of the fluids, as would be understood by one skilled in the art.

It is further believed that after contacting one surface of the porous barrier with the solution and the opposite surface of the interface media with the fresh nutrient media, a liquid-liquid interface is formed between the first sperm sample and the nutrient media within one or more holes in the porous barrier. In some embodiments, the addition of the nutrient media is controlled in a manner that prevents or substantially prevents migration of the nutrient media to the opposite side of the porous barrier, prevents or substantially prevents migration of the solution to the nutrient media side of the porous barrier, or a combination thereof.

Without wishing to be bound by any particular theory, it is believed the sperm having higher motile activity are attracted by the nutrient-containing media more strongly than sperm having lesser motile activity or in combination or alternatively, the motile sperm that are more active are more probable to swim into the fresh, nutrient-containing media Higher motile activity with respect to a single sperm cell means that the sperm cell is more robust in motility and/or shape or the like in comparison to other sperm cells. In a collection of sperm cells, vitality is measured as percent motile population, a percent normal morphology population, or a combination thereof. Being more strongly attracted to the nutrient-containing media, a sperm cell having a higher vitality travela through a hole in the porous barrier, traversing the liquid-liquid interface between the first sperm sample and the nutrient-containing media to then reside in the nutrient-containing media.

With the passage of time, additional sperm cells having higher viability travel through the porous barrier such that the nutrient-containing media side of the porous barrier will comprise nutrient-containing media and a collection of motile sperm cells (“enriched sperm”). The enriched sperm will comprise primarily or only motile sperm cells having higher average vitality than the average vitality of the sperm cells in the first sperm sample prior to addition of the nutrient-containing media to the opposite side of the porous barrier from the semen sample. Sperm cells that remain on the side of the porous barrier with the semen sample (“depleted sperm”) after the enriched sperm travel to the nutrient media will have a lower average vitality than the total sperm cell population in the semen sample prior to addition of the nutrient-containing-media to the opposite side of the porous barrier from the semen sample.

In some embodiments, the porous barrier is contacted with the nutrient-containing media first, then the first sperm sample is added to the opposite side of the porous barrier. In some embodiments, the semen sample is below the porous barrier and the nutrient-containing media is above the porous barrier. In some embodiments, the semen sample is above the porous barrier and the nutrient-containing media is below the porous barrier. In some embodiments, the semen sample and the nutrient-containing media are side-by-side with the porous barrier in between. In some embodiments, regardless of orientation, the porous barrier maintains or substantially maintains an interface or connection between the semen sample and the nutrient-containing media on the opposite side of the porous barrier.

At a time after accumulation of a portion of enriched sperm in the nutrient-containing media or fresh media, a portion of the mixture of enriched sperm and nutrient-containing media having a first concentration of enriched sperm is recovered for use in fertilization of one or more eggs. In some embodiments, the method disclosed herein provides for recovery of enriched sperm without the use of centrifugation, thereby eliminating potential damage to sperm cells resulting from centrifugation.

In some embodiments, the recovered mixture is centrifuged to produce a new mixture of enriched sperm and nutrient-containing media having a second concentration of enriched sperm is recovered for use in fertilization of one or more eggs, wherein the second concentration is greater than the first concentration. In some embodiments, the new mixture has a motile sperm population greater than the motile sperm population of the recovered mixture, a percent normal morphology greater than the percent normal morphology of the recovered mixture, or a combination thereof.

Without wishing to be bound by any particular theory, it is believed that withdrawal of the mixture of nutrient-containing media and enriched sperm to soon after addition of the nutrient-containing media would result in insufficient concentration of the enriched sperm in the nutrient-containing media, and/or withdrawal of the mixture of nutrient-containing media and enriched sperm after too much time would result in too many sperm cells having lesser viability traveling through the porous barrier to the nutrient-containing media. In some embodiments the mixture is recovered at least 15 minutes, at least 30 minutes, or at least 45 minutes after addition of the nutrient media to the surface of the porous barrier. In some embodiments the mixture is recovered no more than 180 minutes, no more than 120 minutes, no more than 90 minutes, or no more than 60 minutes after addition of the nutrient-containing media to the surface of the porous barrier. In some embodiments the mixture is recovered a time period after addition of the nutrient-containing media to the surface of the porous barrier in the range of from 10 minutes to 180 minutes, from 15 minutes to 120 minutes, from 30 minutes to 90 minutes, or from 45 minutes to 60 minutes, after.

The enriched sperm has a motile sperm population greater than the first sperm sample prior to addition of the nutrient-containing media to the opposite side of the porous barrier, a normal morphology sperm population greater than the first sperm sample prior to addition of the nutrient-containing media to the opposite side of the porous barrier, or a combination thereof. In some embodiments, the ratio of the motile sperm population of the enriched sperm to the motile sperm population of the first sperm sample prior to addition of the nutrient-containing media to the opposite side of the porous barrier is greater than or equal to 1.5, greater than or equal to 1.75, or greater than or equal to 2.0. In some embodiments, the ratio of the normal morphology sperm population of the enriched sperm to the normal morphology sperm population of the first sperm sample prior to addition of the nutrient-containing media to the opposite side of the porous barrier is greater than or equal to 1.5, greater than or equal to 1.75, or greater than or equal to 2.0.

In some embodiments, the enriched sperm has motility of greater than or equal to 50%, greater than or equal to 60%, or greater than or equal to 70%, wherein the sperm population in the original semen sample has motility in the range of from 20% to 30%. In some embodiments, the enriched sperm has a normal morphology sperm population of greater than or equal to 50%, greater than or equal to 60%, or greater than or equal to 70%. In some embodiments, the enriched sperm has motility of greater than or equal to 50% and a normal morphology sperm population of greater than or equal to 50%, motility of greater than or equal to 60% and a normal morphology sperm population of greater than or equal to 60%, or motility of greater than or equal to 70% and a normal morphology sperm population of greater than or equal to 70%.

In some embodiments, the porous barrier comprises a plurality of holes, wherein each hole provides a fluid connection between one side of the porous barrier and the opposite side of the porous barrier, and each opening has a passthrough dimension or hydraulic diameter in the range of from 6 microns to 30, preferably 7 microns to 27 microns, or more preferably 7 microns to 25 microns. In some embodiments, a first surface of the porous barrier is in contact with the first sperm sample, and the ratio of the passthrough dimension or hydraulic diameters of holes in the first surface to the passthrough dimension or hydraulic diameters, respectively, of the holes on the second (opposite) surface is greater than or equal to 1.05, greater than or equal to 1.10, or greater than or equal to 1.15. In some embodiments, the combined area of the hole openings on the first surface (in this instance the surface contacting the first sperm sample) is in the range of from 10% to 60%, from 20% to 50%, or from 30% to 40% of the total area of the first surface of the porous barrier. In some embodiments, the porous barrier has a thickness between the first and second surfaces in the range of from 10 microns to 200 microns, from 10 microns to 100 microns, from 15 microns to 80 microns, from 20 microns to 60 microns, or from 25 microns to 50 microns.

In some embodiments, the nutrient-containing or fresh media employed to attract sperm cells having higher viability comprises a zwitterionic organic chemical buffering agent. In some embodiments, the zwitterionic organic chemical buffering agent comprises 4-(2-hydroxyethyl)-1-piperazineethanesulfonic acid. The media mixed with a semen sample and the media on the opposite side of the porous barrier can be nutrient-containing, buffered, or a combination thereof, and can also be the same as or different from each other. It is highly likely that those who choose to process semen samples in an incubator will use a different media with a different set of buffers than the media used at the time of collecting the semen sample (bicarbonate vs. HEPES buffer). In some embodiments, a buffer requires CO₂ to maintain the correct pH. HEPES buffer does not require CO₂, but media used for embryos, which could be used in SID and could be preferable in some situations, requires CO₂.

In some embodiments, the rate of temperature change of the semen sample, the second solution, or a combination thereof, is less than or equal to 0.5° F. (0.28° C.) per minute during the time period after the semen sample and the nutrient media are both in contact with the porous barrier. In some embodiments, temperature change of the semen sample or the second solution is limited by placement of the apparatus (i.e., first vessel and SID) in a temperature-controlled enclosure, wrapping the apparatus with and insulating material, or in the case of the Type 2 apparatus, including insulating material between the sample container and second vessel in the Type 2 embodiments, or in the Type 1 embodiment, surrounding the first vessel and the SID

In some embodiments, a method is disclosed for extracting a second sperm population from a semen sample comprising a first sperm population, wherein the second sperm population has improved vitality for extraction and use in in-vitro fertilization or testing as compared to the first sperm population. The method comprises the steps of: a) obtaining semen sample comprising a first sperm population; b) contacting a nutrient-containing media with the other side of the porous barrier; c) contacting the porous barrier with the semen sample; and d) extracting a second sperm population having improved vitality from the nutrient-containing media. In some embodiments, the method is further described by one or more of the following:
1) step d) extracting the second sperm population occurs at least 15 minutes, at least 30 minutes, or at least 45 minutes after step b) introducing the media;
2) step b) introducing the media and step d) extracting the second sperm population are performed while limiting temperature change of the first and second sperm population to less than or equal to 0.5° F. (0.28° C.) per minute;
3) the porous barrier comprises holes, and each hole has a passthrough dimension or hydraulic diameter in the range of from 6 microns to 35 microns, preferably 7 microns to 30 microns;
4) the contacting in step d) in a manner that substantially or does not, break the surface tension of a meniscus of the first sperm sample; and
5) the method is performed without agitation, manipulation or other physical or chemical enhanced interaction in any of steps a) through d).

### Single-Vessel Embodiments (Type 1 Apparatus)

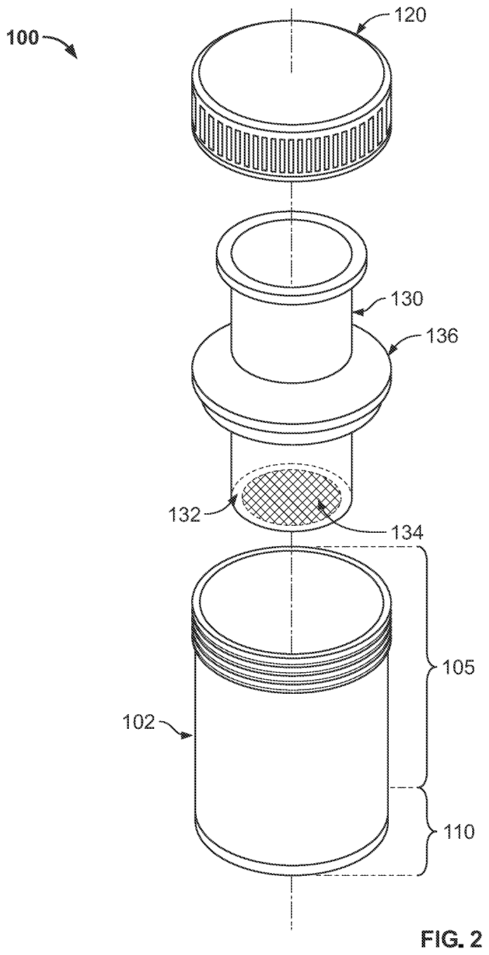

In some embodiments, as shown in FIG. 1-FIG. 5, the apparatus 100 comprises a single vessel. FIG. 1 and FIG. 2 show different views of the same apparatus 100 and accordingly use common reference numbers for common components and portions of the apparatus 100. FIG. 1 shows a vertical cross-section view of apparatus 100 comprising a first vessel 102, or sample container, and related components. FIG. 2 shows a corresponding three-dimensional expanded view of apparatus 100 comprising vessel 102, or sample container, and its related components. The sample container 102 comprises at its upper end collection means 105 and at its lower end a reservoir 110. The single vessel 102 in these embodiments is referred to elsewhere in this disclosure and in the claims as a first vessel or a sample container.

The collection means 105 as shown in FIG. 1 is essentially a vertical wall, which is cylindrical in shape. However, the wall of the collection means 105, when viewed as a vertical cross-section of the vessel can be straight, curved, sloped, or any combination thereof. When liquid, (i.e, first semen sample optionally in a buffered media) is added to the vessel from above, the shape of the wall of the collection means 105 will either guide the flow of liquid by gravity or alternatively not restrict the flow by gravity of liquid toward the reservoir 110.

When viewed as a horizontal cross-section, the wall of the collection means 105 can be circular, oval-shaped, substantially square, substantially rectangular, irregularly shaped, or any other shape as can conveniently meet user preferences for ease of manufacturing, handling, and or storage of the apparatus. The horizontal cross-section can also be variable provided that the wall of the collection means 105 guides fluids entering the top of the vessel by gravity flow toward the reservoir or does not restrict flow of fluids entering the top of the vessel toward the reservoir 110.

The reservoir 110 has a volume suited for the amount of fluid to be collected in the apparatus. The volume for collection of semen is based on the amount of ejaculate from the species from which the semen will be collected, whether human or other mammalian animal.

In some embodiments, in addition to the above aspects of the physical configuration of the apparatus, a closure means or lid 120 is provided to eliminate the possibility of spillage. The lid 120 is shown in FIG. 2 but not in FIG. 1. Lid 120 would be secured to the upper end of vessel 102 during storage and/or transportation of a collected sperm sample but is removed as shown in FIG. 1 for practice of the method disclosed herein.

FIG. 1 further shows lid 120 removed and the SID 130, comprising frame and porous barrier 134, is inserted into sample container 102 for practice of the method disclosed herein. The SID 130 is slidably engaged with a support assembly 136, which is also engaged with sample container 102. The SID 130 can be adjusted vertically such that the porous barrier 134 is in contact with or submerged below the surface of the solution comprising a semen sample 140, optionally further comprising a portion of a media. Nutrient-containing media 142 is shown above the porous barrier 134. In some embodiments, the SID 130 assembly further comprises a flow diverter 132, which functions to keep the nutrient-containing media 142 and the solution comprising a semen sample 140 on their respective sides of the porous barrier 134 as the nutrient-containing media 142 is added to the SID 130 chamber. The flow diverter 132 can be any flat solid surface coextensive with the porous barrier 134 and also integral with the frame. The flow diverter 132 absorbs any component of velocity of the nutrient-containing media 142 is added to the SID 130 and causes the nutrient-containing media to be distributed across the porous barrier 134 by flow tangential to the surface of the porous barrier 134. Flow diverter 132 is shown in FIG. 1 as a flat ring connected to the lower end of the SID 130, but flow diverter 132 could also be a chord shape lip connected to the lower end of SID 130 on just one side of the porous barrier 134 or alternatively a circular shaped disc near or at the center of the porous barrier 134. In the case of a concave or convex shaped porous barrier 134, the flow diverter 132 would follow the contour of the surface of the porous barrier 134 at the chosen installation location such that flow diverter 132 absorbs any component of velocity of the nutrient-containing media 142 is added to the SID 130 and causes the nutrient-containing media to be distributed across the porous barrier 134 by flow tangential to the surface of the porous barrier 134.

In some embodiments, the method comprises providing a Type 1 apparatus 100 as shown in FIG. 1, wherein reservoir 110 contains a solution comprising a semen sample 140, optionally mixed with a portion of a media. Lid 120 (not shown in FIG. 1) is removed from sample container 102 (first vessel) in preparation for insertion of the SID 130 into sample container 102. The SID 130 is slidably attached to SID 130 support 136 with a friction fit weak enough to allow easy movement of SID 130 relative to SID 130 support 136 but strong enough to prevent movement of SID 130 relative to SID support 136 while performing the steps of the method leading up to collection of a new, enhanced, motile sperm sample comprising nutrient-containing media 142 and enriched sperm. The assembly comprising the SID 130 and SID support 136 starts in the retracted position, which means relative positioning of the SID 130 and SID support 136 such that when SID support 136 is fully engaged with the collection means 105, interface media 134 is still a distance from the surface of solution comprising a semen sample 140. The SID 130 is then slid through SID support 136 until interface media 134 contacts, touches, or is submerged below the surface of solution comprising a semen sample 140. Nutrient-containing media 142 is then added to the side of the porous barrier 134 opposite the solution comprising the semen sample 140, optionally impinging on flow diverter 132 prior to contacting the porous barrier 134. After addition of the nutrient-containing media 142, the apparatus 100 is allowed to stand undisturbed for a desired time period, optionally in a temperature-controlled environment. At the end of this time period, the upper side of porous barrier 134 will contain a mixture of nutrient-containing media 142 and enriched sperm. A portion of this mixture is then recovered for further refinement, such as by centrifugation, or for use in fertilization processes. In a preferred embodiment, no centrifugation takes place throughout the method, and/or agitation, of the apparatus throughout the method.

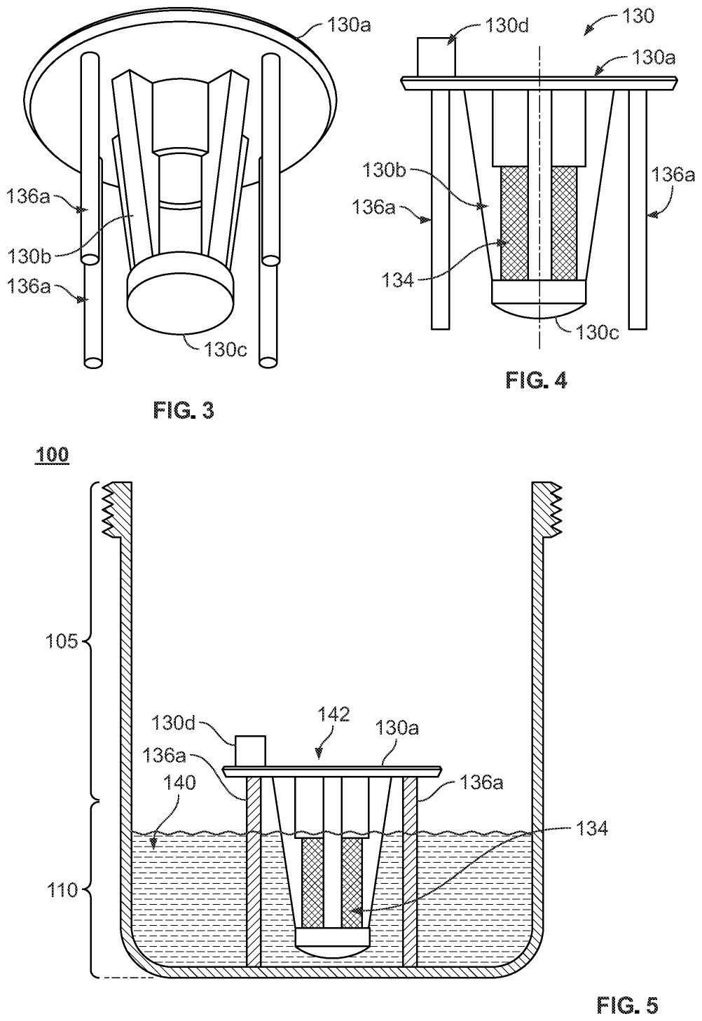

FIG. 3-FIG. 5 show another embodiment of apparatus 100 comprising a single vessel and an alternative configuration of SID 130. FIG. 3 shows a frame, comprising a ring member 130a connected to solid base 130c by downwardly extending supports 130b. A support assembly comprises legs 136a extending downwardly from ring member 130a. The length of legs 136a is such that the SID is supported in the vertical orientation on a flat horizontal surface either with the solid base 130c touching the flat horizontal surface or held a distance above the flat horizontal surface. FIG. 4 shows the porous barrier 134 positioned between each pair of adjacent supports 130b and extending from the solid base 130c to the rim member 130a. SID 130 further comprises lifting handle 130d. FIG. 5 shows a vertical cross-section view of the apparatus 100 comprising a first vessel 102 and related components. The first vessel 102 comprises at its upper end collection means 105 and at its lower end a reservoir 110. The single vessel 102 in these embodiments is referred to elsewhere in this disclosure and in the claims as a first vessel or sample container.

The collection means 105 as shown in FIG. 5 is essentially a vertical wall, which is cylindrical in shape. However, the wall of the collection means 105, when viewed as a vertical cross-section of the vessel can be straight, curved, sloped, or any combination thereof. When liquid is added to the vessel from above, the shape of the wall of the collection means 105 will either guide the flow of liquid by gravity or alternatively not restrict the flow by gravity of liquid toward the reservoir 110.

When viewed as a horizontal cross-section, the wall of the collection means can be circular, oval-shaped, substantially square, substantially rectangular, irregularly shaped, or any other shape as can conveniently meet user preferences for ease of manufacturing, handling, and or storage of the apparatus. The horizontal cross-section can also be variable provided that the wall of the collection means guides fluids entering the top of the vessel by gravity flow toward the reservoir or does not restrict flow of fluids entering the top of the vessel toward the reservoir.

The reservoir 110 has a volume suited for the amount of fluid to be collected in the apparatus. The volume for collection of semen is based on the amount of ejaculate from the species from which the semen will be collected, whether human or other mammalian animal.

In some embodiments, in addition to the above aspects of the physical configuration of the apparatus, a closure means or lid 120 is provided to eliminate the possibility of spillage. The lid 120 is not shown in FIG. 5. Lid 120 would be secured to the upper end of vessel 102 during storage and/or transportation of a collected sperm sample but is removed as shown in FIG. 5 for practice of the method disclosed herein.

FIG. 5 further shows lid 120 removed and the SID 130, comprising a frame and a porous barrier 134, is inserted into vessel 102 for practice of the method disclosed herein. The SID 130 is engaged with sample container 102 (first vessel) by the support assembly comprising legs 136a, which is also engaged with vessel 102. Vertical adjustment of the SID 130 to the surface of the solution 140 is accomplished by managing the depth of the solution in reservoir 105, such that all or at least a portion of porous barrier 134 is submerged in the solution 140. Nutrient-containing media 142 is shown above the porous barrier 134.

In some embodiments, the method comprises providing a Type 1 apparatus 100 as shown in FIG. 5, wherein reservoir 110 contains a solution comprising a semen sample 140, optionally mixed with a portion of a media. Lid 120 (not shown in FIG. 5) is removed from vessel 102 in preparation for insertion of the SID 130 into vessel 102. The SID is inserted into the solution 140 until the porous barrier 134 is partially or fully submerged. Nutrient-containing media 142 is added to the SID chamber either before or after inserting the SID into the solution 140. After the nutrient-containing media 142 and the solution 140 are both in contact with the porous barrier 134, the apparatus 100 is allowed to stand undisturbed for a desired time period, optionally in a temperature-controlled environment. At the end of this time period, the upper side of porous barrier 134 will contain a mixture of nutrient-containing media 142 and enriched sperm. A portion of this mixture is then recovered for further refinement, such as by centrifugation, or for use in fertilization processes, optionally the recovered mixture is used as is for either testing or directly for use, optionally with fresh media, in invitro fertilization.

### Two-Vessel Embodiments (Type 2 Apparatus)

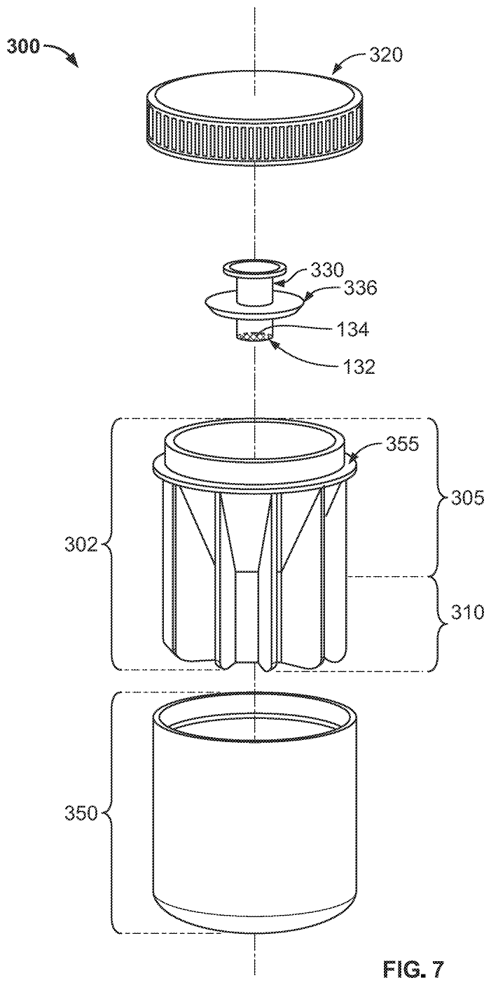

In some embodiments, as shown in FIG. 6-FIG. 10, the apparatus 300 comprises a first and a second vessel. FIG. 6 and FIG. 7 show different views of the same apparatus 300 and accordingly use common reference numbers for common components and portions of the apparatus 300. FIG. 6 shows a vertical cross-section view of the apparatus 300 comprising vessel 302 and related components. FIG. 7 shows a corresponding three-dimensional expanded view of apparatus 300 comprising sample container 302 (first vessel) and its related components. The sample container 302 comprises at its upper end collection means 305 and at its lower end a reservoir 310. The sample container 302 in these embodiments is referred to elsewhere in this disclosure and in the claims as a first vessel or sample container. Sample container 302 and vessel 350 are sized and shaped to allow sample container 302 to be inserted into vessel 350. The vessel 350 in these embodiments is referred to elsewhere in this disclosure and in the claims as a second vessel.

The collection means 305 as shown in FIG. 6 and FIG. 7 is essentially a sloped wall, which is conical in shape (i.e., a funnel). However, the wall of the collection means 305, when viewed as a vertical cross-section of the vessel can be straight, curved, sloped, or any combination thereof. When liquid is added to the vessel from above, the shape of the wall of the collection means 305 will either guide the flow of liquid by gravity or alternatively not restrict the flow by gravity of liquid toward the reservoir 310.

When viewed as a horizontal cross-section, the wall of the collection means 305 can be circular, oval-shaped, substantially square, substantially rectangular, irregularly shaped, or any other shape as can conveniently meet user preferences for ease of manufacturing, handling, and or storage of the apparatus. The horizontal cross-section can also be variable provided that the wall of the collection means 305 guides fluids entering the top of the vessel by gravity flow toward the reservoir 310 or does not restrict flow of fluids entering the top of the vessel toward the reservoir 310.

The reservoir 310 has a volume suited for the amount of fluid to be collected in the apparatus. The volume for collection of semen is based on the amount of ejaculate from the species from which the semen sample will be collected, whether human or other mammalian animal.

In some embodiments, in addition to the above aspects of the physical configuration of the apparatus, a closure means or lid 320 is provided to eliminate the possibility of spillage. The lid 320 is shown in FIG. 7 but not in FIG. 6. Lid 320 would be secured to the upper end of sample container 302 during storage and/or transportation of a collected sperm sample but is removed as shown in FIG. 6 for practice of the method disclosed herein.

FIG. 6 further shows lid 320 removed and the SID 330, comprising frame and porous barrier 334, is inserted into sample container 302 for practice of the method disclosed herein. The SID and support 336 assembly engaged with sample container 302 with the porous barrier in contact with a solution comprising a semen sample 340, optionally mixed with a portion of nutrient media. Nutrient-containing media 342 is shown above the porous barrier 334. In some embodiments, the SID assembly further comprises a flow diverter 332, which functions to keep the nutrient-containing media 342 and the solution comprising a semen sample 340 on their respective sides of the porous barrier 334 as the nutrient media 342 is added. The flow diverter 332 can be any flat solid surface coextensive with the nutrient media side of the porous barrier 334. The flow diverter 332 absorbs any component of velocity of the nutrient media 342 is added to the SID and causes the nutrient media to be distributed across the porous barrier 334 by flow tangential to the surface of the porous barrier 334. Flow diverter 332 is shown in FIG. 6 as a flat ring connected to the lower end of the SID, but flow diverter 332 could also be a chord shape lip connected to the lower end of SID on just one side of the porous barrier 334 or alternatively a circular shaped disc near or at the center of the porous barrier 334. In the case of a concave or convex shaped porous barrier 334, the flow diverter 332 would follow the contour of the surface of the porous barrier 334 at the chosen installation location such that flow diverter 332 absorbs any component of velocity of the nutrient media 342 is added to the SID and causes the nutrient media to be distributed across the porous barrier 334 by flow tangential to the surface of the porous barrier 334.

In some embodiments, the method comprises providing a Type 2 sample container 302 as shown in FIG. 6, wherein reservoir 330 contains a solution comprising a semen sample 340, optionally mixed with a portion of a media, i.e., a buffered media. Lid 320 (not shown in FIG. 6) is removed from sample container 302 in preparation for insertion of the SID into sample container 302. The SID is slidably attached to SID support 336 with a friction fit weak enough to allow easy movement of SID relative to SID support 336 but strong enough to prevent movement of SID relative to SID support 336 while performing the steps of the method leading up to collection of new sperm sample comprising nutrient media 342 and enriched sperm. The assembly comprising the SID and SID support 336 starts in the retracted position, which means relative positioning of the SID and SID support 336 such that when SID support 336 is fully engaged with the collection means 305, interface media 334 is still a distance from the surface of solution comprising a semen sample 340. The SID is then slid through SID support 336 until interface media 334 contacts the surface of solution comprising a semen sample 340. Nutrient-containing media 342 is then added to the side of the porous barrier 334 opposite the solution comprising a semen sample 340, optionally impinging on flow diverter 332 prior to contacting the porous barrier 334. After addition of the nutrient-containing media 342, the apparatus 300 is allowed to stand undisturbed for a desired time period, optionally in a temperature-controlled environment. At the end of this time period, the upper side of porous barrier 334 will contain a mixture of the nutrient-containing media 342 and enriched sperm. A portion of this mixture is then recovered for further refinement, such as by centrifugation, or for use in fertilization processes or, optionally the recovered mixture is used as is for either testing or directly for use, optionally with fresh media, in invitro fertilization.

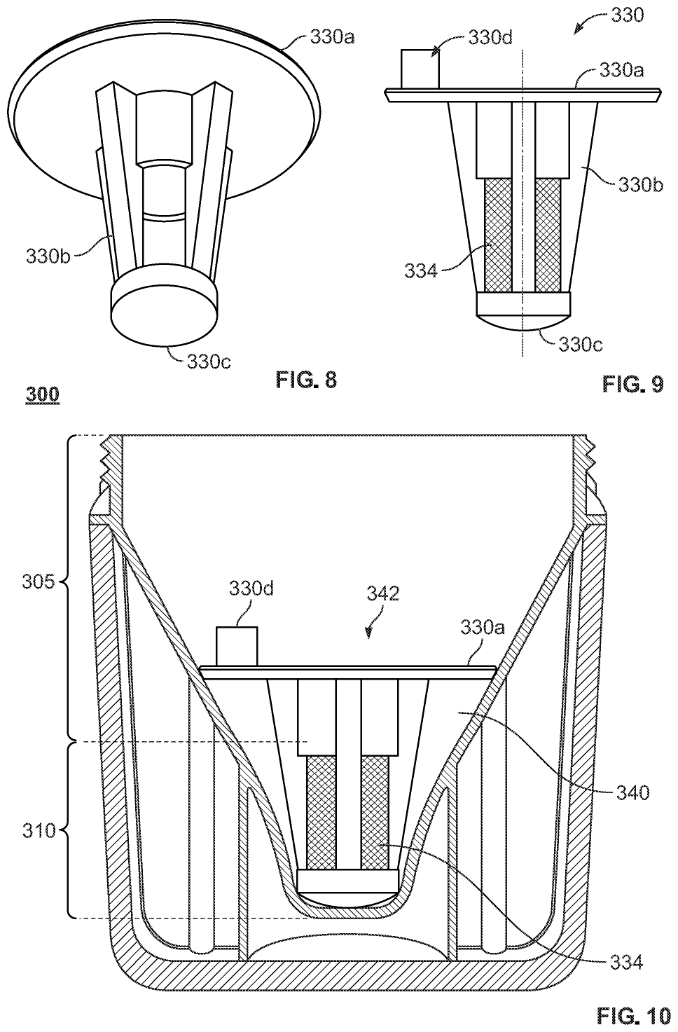

FIG. 8-FIG. 10 show another embodiment of apparatus 200 comprising a first and a second vessel and an alternative configuration of an SID. FIG. 8 shows a frame, comprising a ring member 330a connected to solid base 330c by downwardly extending supports 330b. The length of legs 336a is such that the SID is supported in the vertical orientation on a flat horizontal surface either with the solid base 330c touching the flat horizontal surface or held a distance above the flat horizontal surface. FIG. 9 shows the porous barrier 334 positioned between each pair of adjacent supports 330b and extending from the solid base 330c to the rim member 330a. SID 330 further comprises lifting handle 330d. FIG. 5 shows a vertical cross-section view of the apparatus 300 comprising a sample container 302 (first vessel) and related components. The sample container 302 comprises at its upper end collection means 305 and at its lower end a reservoir 310. The sample container 302 in these embodiments is referred to elsewhere in this disclosure and in the claims as a first vessel. sample container 302 and vessel 350 are sized and shaped to allow sample container 302 to be inserted into vessel 350. The vessel 350 in these embodiments is referred to elsewhere in this disclosure and in the claims as a second vessel.

The collection means 305 as shown in FIG. 10 is essentially a sloped wall, which is conical in shape (i.e., a funnel). However, the wall of the collection means 305, when viewed as a vertical cross-section of the vessel can be straight, curved, sloped, or any combination thereof. When liquid, a bodily fluid, is added to the vessel from above, the shape of the wall of the collection means 305 will either guide the flow of liquid by gravity or alternatively not restrict the flow by gravity of liquid toward the reservoir 310.

When viewed as a horizontal cross-section, the wall of the collection means can be circular, oval-shaped, substantially square, substantially rectangular, irregularly shaped, or any other shape as can conveniently meet user preferences for ease of manufacturing, handling, and or storage of the apparatus. The horizontal cross-section can also be variable provided that the wall of the collection means guides fluids entering the top of the vessel by gravity flow toward the reservoir or does not restrict flow of fluids entering the top of the vessel toward the reservoir.

The reservoir 310 has a volume suited for the amount of fluid to be collected in the apparatus. The volume for collection of semen is based on the amount of ejaculate from the species from which the semen will be collected, whether human or other mammalian animal.

In some embodiments, in addition to the above aspects of the physical configuration of the apparatus, a closure means or lid 320 is provided to eliminate the possibility of spillage. The lid 320 is not shown in FIG. 10. Lid 320 would be secured to the upper end of vessel 320 during storage and/or transportation of a collected sperm sample but is removed as shown in FIG. 10 for practice of the method disclosed herein.

FIG. 10 further shows lid 320 removed and SID, comprising frame and porous barrier 334, inserted into sample container 302 for practice of the method disclosed herein. The SID is engaged with sample container 302 by ring member 330a, which also functions as the support assembly. Vertical adjustment of the SID to the surface of the solution 340 is accomplished by managing the depth of the solution in reservoir 305, such that all or at least a portion of porous barrier 334 is submerged, or partially submerged, in the solution 340. Nutrient-containing media 342 is shown above the porous barrier 334.

In some embodiments, the method comprises providing a Type 1 apparatus 200 as shown in FIG. 10, wherein reservoir 310 contains a solution comprising a semen sample 340, optionally mixed with a portion of a media, i.e., a buffered media Lid 320 (not shown in FIG. 10) is removed from sample container 302 in preparation for insertion of the SID into sample container 302. The SID is inserted into the solution 340 until the interface media 334 is partially or fully submerged. Nutrient-containing media 342 is added to the SID chamber either before or after inserting the SID into the solution 340. After the nutrient-containing media 342 and the solution 340 are both in contact with or touching the porous barrier 334, the apparatus 200 is allowed to stand undisturbed for a desired time period, optionally in a temperature-controlled environment. At the end of this time period, the upper side of porous barrier 334 will contain a mixture of nutrient media 342 and enriched sperm. A portion of this mixture is then recovered for further refinement, such as by centrifugation, or for use in fertilization processes, or for use in fertilization processes, optionally the recovered mixture is used as is for either testing or directly for use, optionally with fresh media, in invitro fertilization.

### Stand-alone SID Embodiments

In some embodiments, a SID as shown in FIG. 3 and FIG. 4 can be utilized as an apparatus independently from a container or apparatus containing a solution comprising a semen sample. A SID 130 for passively isolating a population of motile sperm from a solution comprising a semen sample is disclosed herein. In some embodiments, the SID 130 comprises a frame and a porous barrier 134. In some embodiments, the frame comprises a ring member 130a connected to solid base 130c by downwardly extending supports 130b. The porous barrier 134 comprises a plurality of holes having a passthrough dimension or hydraulic diameter in the range of from 6 microns to 30 microns. In some embodiments, the frame of FIG. 3 and the porous barrier 134 form a chamber having an opening at the upper end suitable for receiving a portion of a media. The SID has a lower portion suitable for insertion into a solution comprising a semen sample. The chamber and the solution are fluidly connected through at least a portion of the holes in a porous barrier when the SID has received the portion of nutrient-containing media, and the SID is inserted into the solution. The SID is adapted for limiting and/or stabilizing the vertical position of the sperm isolation device when inserted into a solution comprising a semen sample. In some embodiments, a support assembly comprises legs 136a extending downwardly from ring member 130a. The length of legs 136a is such that the SID is supported in the vertical orientation on a flat horizontal surface either with the solid base 130c touching the flat horizontal surface or held a distance above the flat horizontal surface.

### SID Assembly

The specific configuration of a SID can vary in order to better integrate and/or function with a Type 1 apparatus or a Type 2 apparatus or as a stand-alone device. The SID comprises a frame and a porous barrier. The frame and porous barrier form a chamber having an opening at its upper end suitable for adding a fluid to the chamber or withdrawing a fluid from the chamber. The frame and the porous barrier can be configured in any manner so long as the chamber of the SID is fluid tight other than the holes in the porous barrier.

In some embodiments, an SID comprises a frame having a ring member attached to a solid base by at least two downwardly extending supports, wherein the porous barrier is positioned between each pair of adjacent supports and extends from the solid base to the rim member to form a chamber suitable for receiving the portion of fluid, such as fresh nutrient media. In such embodiments, the chamber can be cylindrical or frustoconical, wherein at least a portion of the sidewall of the chamber comprises the porous barrier. In such embodiments, in operation of the methods disclosed herein, the SID is partially submerged in a fluid, such as a solution comprising a semen sample. A fresh nutrient media is added to the chamber of the SID before or after contacting the porous barrier with the solution.

In some embodiments, a SID has a cylindrical frame and a porous barrier at its lower end to form a chamber. In some embodiments, a SID has a frame comprising plurality of ring members spaced apart vertically attached to or formed integrally with a plurality of vertical members to form a basket having windows framed at the top and bottom by adjacent ring members and on each side by adjacent vertical members. A porous barrier comprising a pliable mesh can be attached to the outer side of the basket or the inner side of the basket such that the windows are covered with the mesh to form a chamber.

In some embodiments, a SID has a frame formed by in a rigid cup-shaped structure or forming a rigid cup-shaped structure with hole and attached sections of porous barrier to cover each hole to form a chamber.

In some embodiments, a SID has a frame and porous barrier that are integral, such as, but not limited to, forming a rigid cup-shaped structure and perforating one or more regions of the structure such that those regions comprise a porous barrier or alternatively, forming a cup-shaped structure from a porous barrier material that is sufficiently pliable to permit such forming and that is sufficiently rigid so as to be self-supporting.

In some embodiments, the SID comprises a frame having a conical shape, a frustoconical with a flat bottom, a cylindrical shape with a flat bottom, or variations thereof. In such embodiments, one or more porous barriers is disposed on the sides of the frame, one or more porous barriers is disposed on the bottom of the frame, or a combination thereof.

In some embodiments, a porous barrier comprises a plurality of holes, wherein each hole has a passthrough dimension in a range of from 6 microns to 30 microns, from 7 microns to 25 microns, from 8 microns to 20 microns, or from 9 microns to 15 microns. The passthrough dimension is the diameter of the largest sphere that could pass through the hole. In some embodiments, the holes can be straight, curved, and/or branched slots, wherein the width of the slot is the passthrough dimension. In some embodiments, a plurality of slots is arranged parallel to one another, in a radial pattern, or a combination thereof. A slot is a hole having a constant passthrough dimension.

In some embodiments, a porous barrier comprises a plurality of holes, wherein each hole has a passthrough dimension or hydraulic diameter in a range of from 6 microns to 30 microns, from 7 microns to 25 microns, from 8 microns to 20 microns, or from 9 microns to 15 microns. In such embodiments, a plurality of holes is arranged in rows and columns to form a gridwork of holes, such as, but not limited to, a gridwork of holes formed by a fabric mesh. In some embodiments, each hole is the same size. In some embodiments, a plurality of holes comprises holes having a mixture of hydraulic diameters in a range of from 6 microns to 30 microns, from 7 microns to 25 microns, from 8 microns to 20 microns, or from 9 microns to 15 microns. In some embodiments, a plurality of holes comprises holes having a sub range of hydraulic diameters wherein the minimum hole size differs from the maximum hole size by 1 micron, 2 microns, 3 microns, 4 microns, 5 microns, 6 microns, 7 microns, 8 microns, 9 microns, or 10 microns and the subrange is with a range of from 6 microns to 30 microns, from 7 microns to 25 microns, from 8 microns to 20 microns, or from 9 microns to 15 microns.

In some embodiments, a SID comprises one or more porous barriers disposed on the sides of the chamber formed by the frame and one or more porous barriers, wherein the holes and/or slots in the one or more porous barriers form a gradient of sizes of holes and/or slots such that the sizes of the holes and/or slots are increasing or decreasing from the lower part of the SID to the upper part of the SID.

In some embodiments related to a Type 2 apparatus, the wall portion of the SID, the SID support, and the porous barrier are each fabricated using the same or different thermoplastic. In some embodiments, the wall portion of the SID, the SID support, and the porous barrier are each fabricated using the same or different process selected from blow molding, injection molding, and 3D printing.

In some embodiments, the reservoir is marked for adding a predetermined amount of the sperm sample, a predetermined depth for addition of the sperm sample, or a combination thereof. In these embodiments, instead of slidable attachment to a SID support, the SID support is an integral part of the SID and suited to position the porous barrier at the surface of the sperm sample without adjustment. In some embodiments, this is by engagement of the SID support with the collection funnel and/or upper end of the first vessel and/or engagement of one or more legs at the bottom of the SID with the bottom of the reservoir. In further such embodiments, that SID may be tapered, having larger diameter at its upper end, to facilitate easier collection of nutrient media comprising enriched sperm.

In some embodiments, the SID further comprises a guide member attached to the upper end of the SID, wherein the guide member provides a notch, hole, or other stabilizing means for contacting and supporting a transfer device when adding or withdrawing fluid from the chamber of the SID. Such stabilization assists the user in avoiding contact between the transfer device and the porous barrier as such contact may cause breakthrough of the first media into the sample solution and/or the sample solution into the chamber.

In some embodiments, a minimum passthrough dimension or hydraulic diameter of the holes in the porous barrier is selected to provide a passageway of sufficient size to permit motile sperm to swim through the holes. This minimum passthrough dimension or hydraulic diameter will vary according to species as shown below in Table 2.

In some embodiments, a maximum passthrough dimension or hydraulic diameter of the holes in the porous barrier is selected to prevent or reduce the passage of unwanted materials and/or contaminants from passing through the porous barrier and into the second solution. It is believed that a passthrough dimension or hydraulic diameter of less than approximately 50 microns will prevent white blood cells from passing through the porous barrier. It is believed that a passthrough dimension or hydraulic diameter of less than or equal to 30 microns will prevent a variety of unwanted proteins and/or some damaged/fragmented sperm cells from passing through the porous barrier.

In some embodiments, the material from which the porous barrier is formed comprises a static charge, wherein in further embodiments, a specific material of construction might be selected to provide a desired level of static charge. Without wishing to be bound by any particular theory, it is believed that this static charge further inhibits passage of proteins through the porous barrier, even when such proteins are of a size small enough to otherwise pass through the holes having a certain passthrough dimension or hydraulic diameter. Certain proteins have either a positive or negative charge, and it is believed that proteins having an opposite charge from the static charge of the porous barrier will cling to the porous barrier instead of passing through a hole and that proteins having a like charge to the static charge of the porous barrier will be repelled from the porous barrier thus preventing them from entering a hole.

In some embodiments, all materials of construction of the SID (and any other components of the apparatus that may contact the semen sample solution or the second solution) are selected to prevent or minimize damage to sperm cells. In some embodiments, all or a portion of materials of construction of the SID (and any other components of the apparatus that may contact the semen sample solution or the second solution) have surfaces comprising one or more antioxidants to prevent or mitigate oxidative degradation of sperm cells.

### Method Variations

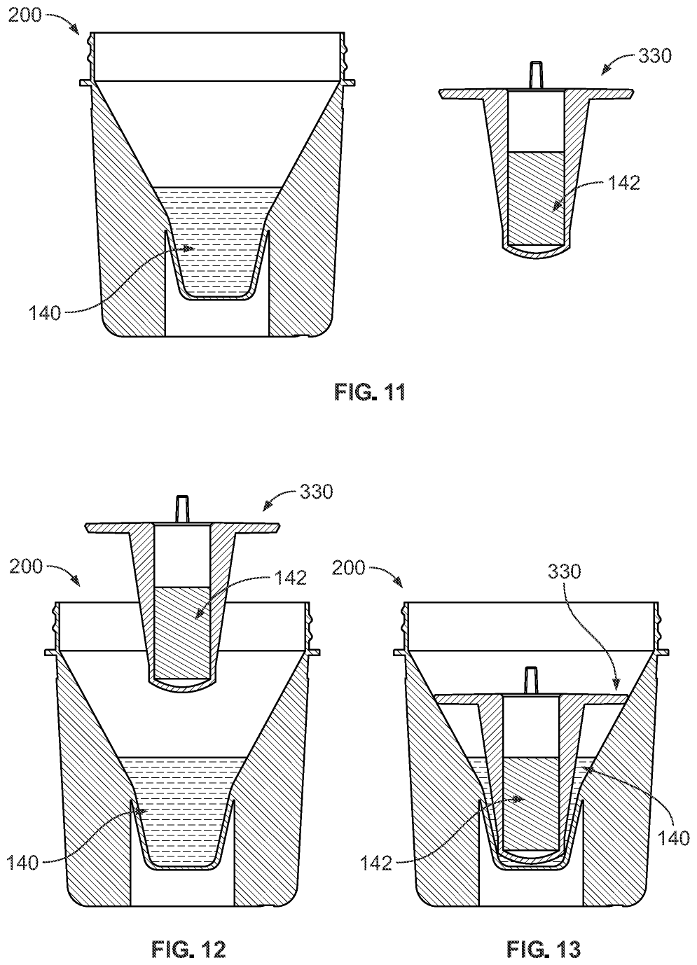

Simplified diagrams are used in FIG. 11-FIG. 13 are used as a benchmark to demonstrate variations of the methods disclosed herein that may be desirable based on individual facts and circumstances. FIG. 11 shows an apparatus 200 containing a solution comprising a semen sample 140 and a SID 330 comprising a porous barrier 334 containing a fresh nutrient-containing media 142. FIG. 12 shows the SID 330 comprising a porous barrier 334 containing a fresh nutrient-containing media 142 being lowered into the apparatus 200 containing a solution comprising a semen sample 140. FIG. 13 shows a SID 330 comprising a porous barrier 334 containing a fresh nutrient-containing media 142 being engaged with the apparatus 200 containing a solution comprising a semen sample 140, such that porous barrier 134 has semen sample 140 on one side and fresh nutrient-containing media 142 on the other side.

In some embodiments, the apparatus 200 contains solution 140, the SID contains nutrient-containing media 142 prior to engaging the apparatus 200 and the SID, and the steps followed are:
1) Solution comprising a semen sample 140 is added to apparatus 200 as shown in FIG. 11.
2) Fresh nutrient-containing media 142 is added to the SID 330 comprising a porous barrier 334 as shown in FIG. 11.
3) The SID 330 is inserted into apparatus 200 as shown in FIG. 12 gently to prevent or minimize any passage of either the nutrient-containing media 142 of the solution 140 through the porous barrier 134 until the SID 330 is engaged with apparatus 200 as shown in FIG. 13.
4) The SID 330 and apparatus 200 remain stationary for a threshold period of time to allow passive migration of motile sperm in the solution 140 into the nutrient-containing media 142. In some embodiments, a threshold period of time is in the range of from 5 minutes to 3 hours, from 10 minutes to 1 hours, or from 15 minutes to 1 hour. In some embodiments, the apparatus 200 is maintained at a temperature in the range of from between and including a range of from 5° C. to 40° C., from 15° C. to 37° C., or from 25° C. to 30° C. In some embodiments, the apparatus 200 remains stationary with no agitation of the solution 140 in the apparatus and/or the nutrient-containing media in the SID 330 or the SID 330 itself
5) After a sufficient time period, an amount of nutrient-containing media 142 comprising a population of motile sperm that have passively, on their own accord, based on their own motility, entered into the nutrient-containing media, is withdrawn from the SID 330 for use in testing, in in vitro fertilization (IVF), in vitro fertilization with intracytoplasmic sperm injection (ICSI), intrauterine insemination (IUI), or other means of artificial insemination without centrifugation.

In some embodiments, the apparatus 200 contains solution 140, nutrient-containing media 142 is added to the SID 330 after engaging the apparatus 200 and the SID 330, and the steps followed are:
1) Solution comprising a semen sample 140 is added to apparatus 200 as shown in FIG. 11.
2) The SID 330 is inserted into apparatus 200 as shown in FIG. 12 prior to the addition of nutrient-containing media to the SID 330. The empty SID 330 is gently inserted into the solution 140 to prevent or minimize any passage of the solution 140 through the porous barrier 134 into the SID chamber through a plurality of holes having a passthrough dimension or a hydraulic diameter of between 6 microns and 30 microns. The SID 330 is lowered until it is engaged with apparatus 200 as shown in FIG. 13.
3) Fresh nutrient-containing media 142 is added to the SID 330 gently to prevent or minimize any passage of either nutrient-containing media 142 of the solution 140 through the porous barrier 134 until the SID 330 is filled with nutrient-containing media 142 to a desired level as shown in FIG. 11.
4) The SID 330 and apparatus 200 remain stationary for a threshold period of time to allow passive migration of motile sperm in the solution 140 into the nutrient-containing media 142. In some embodiments, a threshold period of time is in the range of from 5 minutes to 3 hours, from 10 minutes to 2 hours, or from 15 minutes to 1 hour. In some embodiments, the apparatus 200 is maintained at a temperature in the range of from between and including a range of from 5° C. to 40° C., from 15° C. to 37° C., or from 25° C. to 30° C. In some embodiments, the apparatus 200 remains stationary with no agitation of the solution 140 in the apparatus and/or the nutrient-containing media in the SID 330 or the SID 330 itself.
5) After a sufficient time period, an amount of nutrient-containing media comprising a population of motile sperm is withdrawn from the SID 330 for use in testing, in vitro fertilization (IVF), in vitro fertilization with intracytoplasmic sperm injection (ICSI), intrauterine insemination (IUI), or other means of artificial insemination without centrifugation.

In some embodiments, the nutrient-containing media 142 and solution 140 are reversed versus what is shown in FIG. 11-FIG. 13, such that the apparatus 200 contains nutrient-containing media 142, the SID 330 contains solution 140 prior to engaging the apparatus 200 and the SID 330, and the steps followed are:
1) Instead of solution 140, nutrient-containing media 142 is added to apparatus 200 as shown in FIG. 11.
2) Instead of fresh nutrient media 142, solution 140 is added to the SID 330 comprising a porous barrier 334 as shown in FIG. 11.
3) The SID 330 is inserted into apparatus 200 as shown in FIG. 12 gently to prevent or minimize any passage of either the nutrient-containing media 142 of the solution 140 through the porous barrier 134 until the SID 330 is engaged with apparatus 200 as shown in FIG. 13.
4) The SID 330 and apparatus 200 remain stationary for a threshold period of time to allow passive migration of motile sperm in the solution 140 into the nutrient-containing media 142. In some embodiments, a threshold period of time is in the range of from 5 minutes to 3 hours, from 10 minutes to 2 hours, or from 15 minutes to 1 hour. In some embodiments, the apparatus 200 is maintained at a temperature in the range of from between and including a range of from 5° C. to 40° C., from 15° C. to 37° C., or from 25° C. to 30° C. In some embodiments, the apparatus 200 remains stationary with no agitation of the solution 140 in the apparatus and/or the nutrient-containing media in the SID 330.
5) After a sufficient time period, an amount of nutrient solution 140 comprising a population of motile sperm is withdrawn from the SID chamber for use in testing, in vitro fertilization (IVF), in vitro fertilization with intracytoplasmic sperm injection (IC SI), intrauterine insemination (IUI), or other means of artificial insemination without centrifugation.

In some embodiments, the nutrient-containing media 142 and solution 140 are reversed versus what is shown in FIG. 11-FIG. 13, such that the apparatus 200 contains nutrient-containing media 142, solution 140 is added to the SID 330 after engaging the apparatus 200 and the SID 330, and the steps followed are:
1) Instead of solution 140, nutrient-containing media 142 is added to apparatus 200 as shown in FIG. 11.
2) The SID 330 is inserted into apparatus 200 as shown in FIG. 12 prior to the addition of nutrient-containing media to the SID 330. The empty SID 330 is gently inserted into the nutrient-containing media 142 to prevent or minimize any passage of the nutrient-containing media 142 through the porous barrier 134 into the SID 330. The SID 330 is lowered until it is engaged with apparatus 200 as shown in FIG. 13.
3) Instead of fresh nutrient media 142, solution 140 is added to the SID 330 gently to prevent or minimize any passage of either the nutrient media 142 of the solution 140 through the porous barrier 134 until the SID 330 is filled with nutrient media 142 to a desired level as shown in FIG. 11.
4) The SID 330 and apparatus 200 remain stationary for a threshold period of time to allow passive migration of motile sperm in the solution 140 into the nutrient media 142. In some embodiments, a threshold period of time is in the range of from 5 minutes to 3 hours, from 10 minutes to 2 hours, or from 15 minutes to 1 hour. In some embodiments, the apparatus 200 is maintained at a temperature in the range of from 5° C. to 40° C., from 15° C. to 37° C., or from 25° C. to 30° C. In some embodiments, the apparatus 200 remains stationary with no agitation of the solution 140 in the apparatus and/or the nutrient media in the SID 330.
5) After a sufficient time period, an amount of nutrient solution 140 comprising a population of motile sperm is withdrawn from the SID 330 for use in testing, in vitro fertilization, in vitro fertilization with intracytoplasmic sperm injection (ICSI), intrauterine insemination (IUI), or other means of artificial insemination without centrifugation.

### Species-specific Embodiments

It is known that there are variations in semen and sperm properties among different species. Sperm cell dimensions for various species, including Homo sapiens, are provided in Cummins J M, Woodall P F, On mammalian sperm dimensions, J Reprod Fertil. 1985 September, 75(1):153-75. doi: 10.1530/jrf.0.0750153. PMID: 4032369. Embodiments demonstrated herein have been primarily directed to apparatuses and methods tailored to humans. One skilled in the art would recognize desirable changes to the size of holes in a porous barrier based on the dimensions of sperm cells of non-human, animals relative to those of humans. Semen volumes and sperm concentration in semen also vary widely as shown in Table 1, below. One skilled in the art would further recognize desirable changes to volumes and other dimensions of the first vessel, second vessel, the reservoir or the SID based on these variables using human volumes and concentrations as a benchmark. Table 1 further shows various insemination techniques that may be employed downstream of the methods disclosed herein as well as motivations for and process types that may be employed for various animal species.

TABLE 1

| | Samples | | Techniques | | | | Motivation | | | |
|---|---|---|---|---|---|---|---|---|---|---|
| | Vol. | Conc. | AI | | | | Superior | Reproductive | Processing | |
| Species | (mL) | (billion/mL) | (IUI) | IVF | ICSI | Cryo* | Genetics | Issues | Fresh | Frozen |
| Bovine | 6-10 | 1-1.5 | | X | X | X | X | | partial* | whole |
| Canine | 1-6 | 0.2-0.4 | X | | | X | X | X | whole | whole |
| Equine | 20-100 | 0.03-0.6 | X | X | X | X | X | X | partial* | whole |
| Ovine | 0.75-1.5 | 3.5-5 | X | | | X | X | | partial | whole |
| Porcine | 100-350 | 0.3-0.75 | X | | | X | X | | partial* | whole |
| Gallus | 0.2-0.5 | 4-6 | X | | | X | X | | whole | whole |
| Meleagris | 0.1-0.25 | 8-10 | X | | | X | X | | whole | whole |
| Human | 2-5 | 0.02-0.2 | X | X | X | X | | X | whole | whole |

Sperm from different species are of different sizes and shapes. A porous barrier can be tailored for different species by adapting hole size to accommodate different sperm head dimensions for different species. Table 2 shows sperm head average dimensions for various species. Functional surface area from tip of head (um²) is the hole size required if the sperm is swimming “nose first.” Functional area if from wide side (um²)-sperm swim with head at 90° angle to tail presenting the largest possible surface area to pore or hole. The majority of motile sperm will swim nose first.

TABLE 2

| | | | | Functional | |
|---|---|---|---|---|---|
| | | | | Surface Area | Functional |
| | Length | Width | | from tip of | Area if from |
| Species | (um) | (um) | Shape | head (um²) | wide side (um²) |
| Cattle | 9 | 5 | egg | 7.85 | 9-15 |
| Dog | 5 | 3 | paddle | 4.5 | 10-15 |
| Horse | 6.2 | 3 | egg | 4.71 | 6.2-9.3 |
| Human | 5 | 3 | egg | 4.71 | 5-7.5 |
| Pig | 9 | 4.5 | paddle | 9 | 18-40.5 |
| Sheep | 8.1 | 4.8 | ovoid | 7.54 | 12.96-19.44 |

### Certain Embodiments

Disclosed herein are embodiments of an apparatus for passively isolating a population of motile sperm from a semen sample solution. In some embodiments, the apparatus for passively isolating a population of motile sperm from a semen sample solution comprises:
a) a first vessel for receiving the solution, and
b) a sperm isolation device for receiving a portion of a media, the device comprising a frame and a porous barrier, the porous barrier comprising a plurality of holes having a passthrough dimension or a hydraulic diameter in the range of from 6 microns to 30 microns.

In some embodiments of the apparatus, in addition to the above limitations of the above embodiment, the apparatus is further characterized by the following:
a) the first vessel has a reservoir suitable for receiving the solution;
b) the sperm isolation device has a chamber suitable for receiving the portion of the media;
c) the sperm isolation device fits within the first vessel such that the reservoir and the chamber are fluidly connected through at least a portion of the holes in the porous barrier when:
   i) the first vessel has received the sperm sample;
   ii) the sperm isolation device has received the portion of media; and
   iii) the sperm isolation device is engaged with the first vessel.

In some embodiments of the apparatus, in addition to the limitations of any of the above embodiments, the apparatus is further characterized by one or more of the following:
a) the sperm isolation device is substantially conical and adapted for limiting and stabilizing its insertion within the first vessel;

In some embodiments of the apparatus, in addition to the limitations of any of the above embodiments, the apparatus is further characterized by the following:
a) the frame of the sperm isolation device has a rim member attached to a solid base by at least two downwardly extending supports; and
b) the porous barrier is positioned between each pair of adjacent supports and extends from the solid base to the rim member.

In some embodiments of the apparatus, in addition to the limitations of any of the above embodiments, the apparatus is further characterized by one or more of the following:
a) the apparatus does not include any means for promoting fluid flow between the reservoir in the first vessel and the chamber or SID in the sperm isolation device;
b) the apparatus further comprises a removable cover or seal for the first vessel and/or the SID; and
c) the apparatus comprises more than one sperm isolation device disposed within the first vessel.

Disclosed herein are embodiments of a method for passively isolating a population of motile sperm from a semen sample solution. In some embodiments, the method for passively isolating a population of motile sperm from a semen sample solution comprises:
(i) introducing the semen sample solution into a first vessel;
(ii) introducing a harvest media into a sperm isolation device (SID) comprising a porous barrier; and
(iii) inserting the SID in the semen sample solution to submerge at least a portion of the porous barrier;
wherein:
   the porous barrier comprises a plurality of holes, each hole having a passthrough dimension in a range of from 6 microns to 30 microns; and
   a population of motile sperm in the semen sample solution migrate through the holes in the porous barrier into the first media.

In some embodiments of the method, in addition to the limitations of any of the above embodiments, the method is further characterized by one or more of the following:
a) the semen sample solution further comprises a sample media, and the sample media and the harvest media are the same or different;
b) step (iii) is performed in a manner to prevent or minimize transfer through the porous barrier of the semen sample solution into the SID and/or the harvest media into the semen sample solution;
c) in step (ii) the harvest media is added to the SID via a transfer device, wherein the transfer device does not contact the porous barrier; and
d) migration of the motile sperm through the porous barrier into the harvest media is a result of chemotaxis.

In some embodiments of the method, in addition to the limitations of any of the above embodiments, the method is further characterized by the following:
a) after step (ii) and before step (iii), the semen sample solution has a first concentration of sperm, and the harvest media has no concentration of sperm;
b) at a threshold time period after step (iii), the semen sample solution has a second concentration of sperm, and the harvest media has a third concentration of sperm; and
c) the second concentration is less than the first concentration, wherein in further embodiments, the threshold time period is in the range of from 5 minutes to 3 hours, from 10 minutes to 2 hours, or from 15 minutes to 1 hour.

In some embodiments of the method, in addition to the limitations of any of the above embodiments, the method is further characterized by the following:
a) after step (ii) and before step (iii), the semen sample solution comprises a first population of sperm having a first motility, and the harvest media comprises no sperm;
b) at a threshold time period after step (iii), the semen sample solution comprises a second population of sperm having a second motility, and the harvest media has a third concentration of sperm having a third motility;
c) the second population of sperm is less than the first population of sperm;
d) the third population of sperm is the difference between the second population of sperm and the first population of sperm; and
e) the third motility is greater than the second motility, wherein in further embodiments, the threshold time period is in the range of from 5 minutes to 3 hours, from 10 minutes to 2 hours, or from 15 minutes to 1 hour.

In some embodiments of the method, in addition to the limitations of any of the above embodiments, the method further comprises withdrawing a portion of the harvest media at a second threshold time period after step (iii) to recover a second solution, wherein in further embodiments:
a) the second threshold time period is in the range of from 5 minutes to 3 hours, from 10 minutes to 2 hours, or from 15 minutes to 1 hour;
b) the second solution is used in testing, in vitro fertilization (IVF), in vitro fertilization with intracytoplasmic sperm injection (ICSI), intrauterine insemination (IUI), or other means of artificial insemination without centrifugation; or
c) a combination thereof.

In some embodiments of the method, in addition to the limitations of any of the above embodiments, the method is conducted:
a) at a temperature in the range of from 5° C. to 40° C., from 15° C. to 37° C., or from 25° C. to 30° C.;
b) with no movement of the apparatus in step (iv);
c) with no agitation of the semen sample solution in the first vessel and/or the harvest media in the sperm isolation device; or
d) a combination thereof.

In some embodiments of the method, in addition to the limitations of any of the above embodiments, the method is further characterized by one or more of the following:
a) the holes have in passthrough dimension or a hydraulic diameter in the range of:
   i) from 6 microns to 12 microns, and the semen sample is from a human; or
   ii) from 7 microns to 30 microns, and the semen sample is from an animal;
b) the semen sample solution comprises a volume ratio of the sample media to the semen sample in the range of from 1:5 to 1:1; and
c) volume ratio of the semen sample solution to the fresh media is in the range of from 2:3 to 10:1, from 1:1 to 8:1, or from 2:1 to 6:1.

In some embodiments of the method, in addition to the limitations of any of the above embodiments, the method is further characterized by the following:
a) after step (ii) and before step (iii), the semen sample solution comprises a first population of motile sperm, and the harvest media comprises no sperm;
b) at a threshold time period after step (iii), the semen sample solution comprises a second population of motile sperm, and the harvest media has a third concentration of motile sperm; and
c) the second population of motile sperm is in the range of from 5% to 30% of the first population of motile sperm, wherein in further embodiments, the threshold time period is in the range of from 5 minutes to 3 hours, from 10 minutes to 2 hours, or from 15 minutes to 1 hour.

In some embodiments of the method, in addition to the limitations of any of the above embodiments, step (iii) is performed prior to step (ii).

In some embodiments of the method, in addition to the limitations of any of the above embodiments, the semen sample solution has not been agitated, centrifuged, or otherwise treated prior to step (i).

Disclosed herein are embodiments of a sperm isolation device for passively isolating a population of motile sperm from a semen sample solution. In some embodiments, the sperm isolation device for passively isolating a population of motile sperm from a semen sample solution comprises:
a) a frame; and
b) a porous barrier, comprising a plurality of holes having a passthrough dimension or a hydraulic diameter in the range of from 6 microns to 30 microns.

In some embodiments of the sperm isolation device, in addition to the limitations of the above embodiment, the sperm isolation device is further characterized by the following:
a) the frame and the porous barrier form a chamber having an opening at the upper end suitable for receiving a portion of a media;
b) the sperm isolation device has a lower portion suitable for insertion into a semen sample solution; and
c) the chamber and the semen sample solution are fluidly connected through at least a portion of the holes in the porous barrier when:
   i) the sperm isolation device has received the portion of media; and
   ii) the sperm isolation device is inserted into the semen sample solution.

In some embodiments of the sperm isolation device, in addition to the limitations of any of the above embodiments, the sperm isolation device is further characterized by one or more of the following:
a) the frame of the sperm isolation device is adapted for limiting and/or stabilizing the vertical position of the sperm isolation device when inserted into a semen sample solution;
b) the holes have in passthrough dimension or a hydraulic diameter in the range of:
   i) from 6 microns to 12 microns, and the semen sample is from a human; or
   ii) from 7 microns to 30 microns, and the semen sample is from an animal.

In some embodiments of the sperm isolation device, in addition to the limitations of any of the above embodiments:
a) the frame of the sperm isolation device has a rim member attached to a solid base by at least two downwardly extending supports; and
b) the porous barrier is positioned between each pair of adjacent supports and extends from the solid base to the rim member, and optionally
c) the sperm isolation device further comprises a removable cover or seal that fits within or around the rim member.

Disclosed herein are embodiments of a method for using any of the above embodiments of the sperm isolation device for passively isolating a population of motile sperm from a semen sample solution. In some embodiments, the method comprises:
(i) introducing a media into the chamber of the sperm isolation device;
(ii) inserting the lower portion of the sperm isolation device into a solution comprising a first amount of motile sperm; and
(iii) after a threshold time period, withdrawing a portion of the media in the chamber with a with a transfer device, wherein the media comprises a second amount of motile sperm.

In some embodiments of the method for using the sperm isolation device, in addition to the limitations of any of the above embodiments, step (iii) is performed prior to step (ii).

In some embodiments of the method for using the sperm isolation device, in addition to the limitations of any of the above embodiments, the method further comprises withdrawing a portion of the harvest media at a second threshold time period after step (iii) to recover a second solution, wherein in further embodiments:
a) the second threshold time period is in the range of from 5 minutes to 3 hours, from 10 minutes to 2 hours, or from 15 minutes to 1 hour;
b) the second solution is contacted with an egg for fertilization without centrifugation, wherein in further embodiments, the method for contacting is selected from in vitro fertilization (IVF), in vitro fertilization with intracytoplasmic sperm injection (ICSI), intrauterine insemination (IDI), or other means of artificial insemination; or
c) a combination thereof.

In some embodiments of the method for using the sperm isolation device, in addition to the limitations of any of the above embodiments, step (iii) is performed prior to step (ii).

The method of claim 45, wherein the semen sample was frozen, is less than 1 mL, or a combination thereof.

Disclosed herein are embodiments of a method for passively isolating a population of motile sperm from a semen sample solution. In some embodiments, the method for passively isolating a population of motile sperm from a semen sample solution comprises:
(i) introducing a harvest media into a first vessel;
(ii) introducing the semen sample into a sperm isolation device (SID) comprising a porous barrier, wherein in further embodiments the semen sample was frozen, is less than 1 mL, or a combination thereof; and
(iii) inserting the SID in the solution to submerge at least a portion of the porous barrier; wherein:
   the porous barrier comprises a plurality of holes having a passthrough dimension or a hydraulic diameter in the range of from 6 microns to 30 microns; and
   a population of motile sperm in the semen sample migrate through the porous barrier into the harvest media.

In some embodiments, a method for collection of sperm with improved viability comprises:
a) providing a first sperm sample comprising sperm having a first viability;
b) contacting a first surface of a porous barrier with the first sperm sample;
c) adding a nutrient media to a second surface opposite the first surface of the porous barrier; and
d) producing a second sperm sample comprising the nutrient-containing media and sperm having a second viability.

In further embodiments of the method for collection of sperm with improved viability, the method further comprises:
recovering a portion of the second sperm sample for use in fertilization, wherein the step of recovering the portion of the second sperm sample is at least 15 minutes, at least 30 minutes, or at least 45 minutes after the step of adding the nutrient media to the second surface of the porous barrier; or
a) centrifuging a portion of the second sperm sample; and
b) recovering a third sperm sample comprising the nutrient-containing media and sperm having:
   i) a third motile population greater than the second motile population;
   ii) the third normal morphology greater than the second normal morphology; or
   iii) a combination thereof.

In further embodiments of any of the preceding embodiments of the method for collection of sperm with improved viability, the method can be further characterized by one or more of the following:
a) the second motile sperm population is greater than in the first motile sperm population; the second normal morphology sperm population is greater than in the first normal morphology sperm population; or a combination thereof;
b) the second motile sperm population is greater than or equal to 50%, greater than or equal to 60%, or greater than or equal to 70%; the second normal morphology sperm population is greater than or equal to 50%, greater than or equal to 60%, or greater than or equal to 70%; or a combination thereof;
c) the porous barrier comprises a plurality of holes, wherein each hole provides a fluid connection between an opening on the second surface and an opening on the first surface of the porous barrier, and has a passthrough dimension or a hydraulic diameter in the range of from 7 microns to 35 microns, wherein some embodiments are further characterized by one or more of the following:
   i) contacting the first surface of a porous barrier with the first sperm sample produces a level of the first sperm sample in one or more holes in the range of from above the bottom surface of the porous barrier to no higher than the second surface of the porous barrier;
   ii) for each hole, the ratio of the passthrough dimension or a hydraulic diameter of the opening on the first surface of the porous barrier to the passthrough dimension or a hydraulic diameter, respectively, of the opening on the second surface of the porous barrier is greater than or equal to 1.05, greater than or equal to 1.10, or greater than or equal to 1.15;
   iii) the combined area of the openings on the bottom surface of the porous barrier is in the range of from 10% to 60%, from 20% to 50%, or from 30% to 40% of the total area of bottom surface of the porous barrier; and
   iv) the porous barrier has a thickness between the first and second surfaces of the porous barrier in the range of from 10 microns to 200 microns, from 10 microns to 100 microns, from 15 microns to 80 microns, from 20 microns to 60 microns, or from 25 microns to 50 microns;
d) the ratio of the second motile population to the first motile population is greater than or equal to 1.5;
e) the nutrient media for the semen sample and the nutrient media on the opposite side of the porous barrier can be the same or different; in some embodiments, the nutrient media comprises any buffering agent as is known in the art, in particular bicarbonate, HEPES, or a combination thereof, and in further embodiments, the nutrient media is a zwitterionic organic chemical buffering agent, and in some embodiments, the zwitterionic organic chemical buffering agent comprises 4-(2-hydroxyethyl)-1-piperazineethanesulfonic acid; and
f) the rate of temperature change of the first sperm sample, the second sperm sample, or a combination thereof, is less than or equal to 0.5° F. (0.28° C.) per minute.

In some embodiments, a method for extracting from a specimen of a first sperm population a second sperm population having improved motility for extraction and use in in-vitro fertilization, the method comprising the steps of:
a) obtaining the specimen of a first sperm population;
b) contacting a porous barrier with the specimen;
c) introducing a media to the porous barrier; and
d) extracting a second sperm population having improved motility from the media.

In further embodiments of the method for extracting from a specimen of a first sperm population a second sperm population having improved viability for extraction and use in in-vitro fertilization, the method can be further characterized by one or more of the following:
a) step d) extracting the second sperm population occurs at least 15 minutes, at least 30 minutes, or at least 45 minutes after step c) introducing the media;
b) step c) introducing the media and step d) extracting the second sperm population are performed while limiting temperature change of the first and second sperm population to less than or equal to 0.5° F. (0.28° C.) per minute;
c) the porous barrier comprises holes, and each hole has a passthrough dimension or a hydraulic diameter in the range of from 10 microns to 35 microns; and
d) the contacting does not break the surface tension of the meniscus of the first sperm sample.

In some embodiments, a method for collection of sperm having improved viability, the method comprising:
a) providing an apparatus comprising a first vessel having a collection funnel at the upper end of the first vessel and a reservoir at the lower end of the first vessel;
b) adding a first sample comprising sperm having a first motile population to the reservoir;
c) engaging a SID support with the collection funnel, wherein a SID is slidably connected to the SID support, and the SID has a solid wall and a bottom comprising a porous barrier;
d) contacting the lower surface of a porous barrier with the first sample in the reservoir by sliding the SID within the SID support;
e) adding a nutrient media to SID in an amount sufficient to cover the upper surface of the porous barrier; and
f) producing a second sample comprising the nutrient media and a first concentration of sperm having a second motile population, wherein the second motile population is greater than the first motile population.

In some embodiments, a porous barrier is provided for use in extracting from a specimen of a first sperm population a second sperm population having improved viability, the porous barrier comprising a plurality of holes, each having a passthrough dimension or a hydraulic diameter in the range of from 10 microns to 30 microns, wherein the porous barrier, used in conjunction with a media, is configured to provide for the extraction of the second sperm population from the specimen.

In some embodiments, an apparatus comprises:
a) a first vessel having a reservoir at the lower end of the first vessel, and optionally a second vessel, wherein the first vessel is inserted into the second vessel;
b) a SID having a solid wall and a bottom comprising a porous barrier; and
c) a SID support, configured to position the bottom of the SID within the reservoir.

In further embodiments of the apparatus, the apparatus can be further characterized by one or more of the following:
a) at least a portion of the bottom of the SID is solid;
b) the first vessel further comprises a collection funnel, and the SID support is configured to engage with the collection funnel; and
c) the SID support is slidably connected to the SID to provide vertical adjustment of the porous barrier within the reservoir.

The following examples illustrate the invention; however, those skilled in the art will recognize numerous variations within the spirit of the invention and scope of the claims. To facilitate a better understanding of the present invention, the following examples of preferred embodiments are given. In no way should the following examples be read to limit, or to define, the scope of the invention.

### Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example Set 1

### Starting Materials

Containers

ProteX™ semen collection containers, available from Reproductive Solutions, Inc. in Dallas, Texas, were used for collection of human semen. ProteX™ semen collection containers hereinafter referred to as C1.

Media

In some cases, semen samples are mixed with media to preserve viability and minimize damage to the sperm during collection and storage. The media with which semen samples are mixed can be the same or different from the media added during the process disclosed herein. In these examples, both media were the same.

### Prepared Materials

SID

A first SID was fabricated from thermoplastic using a 3D printer. Nominal dimensions for the cylindrical portion of the SID were approximately 14 mm outside diameter and approximately 25 mm tall, but these dimensions vary according to the container in which the sperm sample is collected. The dimensions of the SID and SID support work in unison to provide for supporting the porous barrier to contact the surface of the sperm sample. The first SID is hereinafter referred to as B1.

### Porous Barrier

The porous barriers in various examples were fabricated from thermoplastic. Pore sizes in various experiments were 10 μm, 20 μm, 30 μm, or 40 μm. The porous barrier can be formed from a sheet with pores added mechanically, by 3D printing, or by being inherent in a mesh material. The first SID is hereinafter referred to as B1 (porous barrier contacts surface of semen sample solution; see FIG. 1, FIG. 2, FIG. 6, and FIG. 7).

In some embodiments, the porous barrier can be formed from a thermoplastic mesh. In some embodiments, the thermoplastic mesh can be characterized by one or more of a mesh opening size in the range of from 5 μm to 40 μm or from 10 μm to 30 μm (wherein the mesh opening can be conveniently converted to a passthrough dimension or a hydraulic diameter), an open area of from 5% to 30% or from 10% to 25%, a mesh count of from 100 n/cm to 300 n/cm or from 140 n/cm to 250 n/cm, a thread diameter of from 30 μm to 40 μm, a weight of from 30 g/m² to 55 g/m² or from 34 g/m² to 42 g/m², and a thickness of from 50 μm to 80 μm or from 60 μm to 75 μm. Exemplary meshes can be obtained from SAATI Americas Corp. as shown in Table 3 below.

TABLE 3

| | | Mesh | Open | Mesh | Thread | | |
|---|---|---|---|---|---|---|---|
| | | Opening | Area | count | diameter | Weight | Thickness |
| Item | Fiber | (μm) | (%) | (n/cm) | (μm) | (g/m²) | (μm) |
| PA 32/20 | PA | 32 | 20 | 140 | 37 | 36 | 68 |
| PA 31/21 | PA | 31 | 21 | 150 | 37 | 40 | 68 |
| PA 25/21 | PA | 25 | 21 | 180 | 30 | 31 | 60 |
| PA 25/15 | PA | 25 | 15 | 165 | 37 | 42 | 75 |
| PA 21/17 | PA | 21 | 17 | 200 | 30 | 34 | 65 |
| PA 18/14 | PA | 18 | 14 | 205 | 30 | 35 | 57 |
| PA 15/10 | PA | 15 | 10 | 200 × 250 | 30 | 41 | 70 |
| PA 10/4 | PA | 10 | 4 | 200 × 220 | 30 × 38 | 50 | 78 |
| PA 7/2 | PA | 7 | 2 | 200 × 230 | 30 × 38 | 52 | 78 |

### Equipment

In some instances, prepared semen samples were analyzed for sperm movement or kinematics using a Hamilton Thorne IVOS computer assisted semen analyzer (“CASA”), available from Hamilton Thorne in Beverly, Massachusetts. (Version 12 software for IVOS)

In some instances, prepared human semen samples were analyzed for DNA integrity using a Halosperm™ G2 test kit, available from Spectrum Technologies in Healdsburg, California and a standard saturated chlortetracycline technique.

### Definitions

When referring to cells/ml of media, cells mean individual spermatozoa cells per milliliter of fluid.

Analysis of variance (“ANOVA”) means a collection of statistical models and their associated estimation procedures (such as the “variation” among and between groups) used to analyze the differences among means.

Amplitude of lateral head or lateral displacement (“ALH”): Displacement corresponding to the mean width of the head oscillation as the sperm swims. Mean ALH is calculated from all cell tracks that have a straightness greater than the threshold STR and are not measured as SLOW. Measured using CASA.

Average pathway velocity or path velocity (“VAP”): Average velocity of the smoothed cell path in microns/second. Slow cells are excluded from the average. Measured using CASA.

Beat cross frequency (“BCF”): Frequency with which the sperm track crosses the sperm path (i.e., frequency of sperm head crossing the sperm average path in either direction). This value is measured in crossings per second (Hertz). A cell must not be slow to be included in the average. Measured using CASA.

Concentration: Sperm count per unit volume, typically million cells/ml (M/ml). Measured using CASA.

Elongation: Average value of the ratio of minor to major axis of all sperm heads.

Linearity (“LIN”): Average value of the ratio VSL/VCL. LIN measures the departure of the cell track from a straight line. Measured using CASA.

Manual volume measurement: Manual measurement entered into CASA.

Medium cells: Fraction of all cells moving with VAP cutoff<VAP<progressive cell VAP.

Mitochondrial intactness: Analyzed using a standard mitotracker red technique.

Morphology: The size and shape of sperm; examined as part of a semen analysis to evaluate male infertility. Sperm morphology results are reported as the percentage of sperm that appear normal when semen is viewed under a microscope. Analyzed using a rapid H&E staining technique.

Motility: Healthy sperm motility is defined as sperm with forward progressions of at least 25 micrometers per second, typically reported as percentage of total sperm that are “motile.” Measured using CASA. Results of a donor are normalized against motility at collection time of that donor's sperm for comparison to other donors.

Progressive cells: Number of cells moving with both VAP>medium VAP cutoff and straightness STR>S₀.

Rapid cells: Percentage of motile cells with VAP>medium VAP cutoff. Measured using CASA. Results of a donor are normalized against rapid cells at collection time of that donor's sperm for comparison to other donors.

Slow cells: Fraction of all cells moving with VAP<VAP cutoff or VSL<VSL cutoff.

Straight-line velocity, progressive velocity, or progression (“VSL”): Average velocity measured in a straight line from the beginning to the end of track. Slow cells are excluded from the average. Measured using CASA.

Straightness (“STR”): Average value of the ratio VSL/VAP. STR measures the departure of the cell path from a straight line. Measured using CASA.

Track speed or curvilinear velocity (“VCL”): average velocity measured over the actual point-to-point track followed by the cell. Slow cells are excluded from the average. Measured using CASA.

### REFERENCES

WHO laboratory manual for the Examination and processing of human semen, Fifth Ed., World Health Organization (“WHO Manual”).

### Experimental Methods

A. Semen Sample Collection and Treatment

For each semen sample in a C1 container, SID B1 was inserted into container C1 such that the porous barrier at the bottom of the SID B1 contacted the surface of the semen sample solution. For each semen sample in a C1 container, SID B1 was inserted into container C1 such that the porous barrier at the bottom of the SID B1 contacted the surface of the semen sample solution.
1. Samples were collected for clinical semen analysis and later transferred to C1 containers.
2. Standard semen parameters were collected via CASA (HamiltonThorn) for original sperm sample.
4. SID B1 was fit into container C1 such that the bottom of the porous barrier contacted the meniscus of the sample or SID B2 was fit into container C1 such that the porous barrier was submerged in the sample
5. 0.25 mL was added inside the SID (upper side of porous barrier)
6. Lid was placed loosely on container C1 to prevent dehydration and limit light
7. Covered containers C1 were allowed to stand at room temperature for 30 minutes
8. Mixture of media and enriched sperm (˜4 μl) was recovered by pipette from SID B1 and placed onto CASA slide
9. Standard semen parameters were collected via CASA (HamiltonThorn) was performed of enriched sperm sample.
Trial 1-2—test tube with 40 um mesh
Trial 3—prototype 10 mesh
Trial 4—prototype 10, 20 and 30 mesh

Early prototype studies were very limited to 1-3 three samples. In three experiments of a prototype B1 SID produced by 3D printing, second solutions containing an average of 88% motile sperm were produced from semen sample solutions containing an average of 39% motile sperm, but all sperm cells were dead after 24 hours due to toxicity of the 3D printing material. In one experiment of a prototype B2 SID having a conical shape, a second solution containing 70% motile sperm was produced from a semen sample solution containing 32% motile sperm. Percentages are motile sperm population as a fraction total sperm population.

### Example Set 2

Starting Materials

### Containers

ProteX™ semen collection containers, available from Reproductive Solutions, Inc. in Dallas, Texas, were used for collection of human semen. ProteX™ semen collection containers hereinafter referred to as C1.

Media

In some cases, semen samples are mixed with media to preserve viability and minimize damage to the sperm during collection and storage. The media with which semen samples are mixed can be the same or different from the media added during the process disclosed herein. In these examples, both media were the same.

SID

A SID as disclosed herein (submerged porous barrier; see FIG. 3-5 and FIGS. 8-10) was used for processing of human semen, and hereinafter are referred to as SID B2.

Experimental Design

To evaluate the C1/B2 system, studies were conducted at two private fertility clinics. Deidentified semen samples were obtained from two clinical laboratory facilities following routine sample collection and evaluation for fertility assessment as allowed by the standard patient treatment consent.

Prior to collection, a sterile C1 was preloaded with a one-milliliter volume of media used for sperm preparation in that clinic. The patient then collected their sample by masturbation and the specimen was assessed clinically using standard lab protocols. Once the clinical semen analysis was complete, the sample was deidentified and handed off for research.

Each sample underwent a second, abbreviated semen analysis consisting of volume, concentration, and motility using a computer-assisted semen analyzer (CASA) to determine eligibility for inclusion in the study. To enter the study, the sample had to meet inclusion criteria of a minimum of two milliliters of raw semen (a total of three mL with 1 mL of additional media), 30 million total cells, and a minimal initial motility of 30%. Samples meeting the inclusion criteria were then split in half, wherein half of the samples were processed with the B1/C2 system, and the other half being processed using a swim-up procedure (control). The swim-up was the only available procedure that would allow for multiple evaluations across the processing time points and a direct comparison of the efficiency of the new device.

Control samples were prepared by transferring half of the sample into a test tube and centrifuging for 10 minutes. Once the sample had been centrifuged, the supernatant was removed, and the pellet of sperm was kept in the bottom of the test tube. Then, 0.75 mL of sperm-washing medium was layered over the sample. The tube was placed in the incubator (37° C.). At times 0, 5, 15, 30, and 60 minutes, a small aliquot was sampled from the top layer of the specimen and underwent semen analysis in the CASA system.

Experimental samples were prepared with the remaining half of the above samples. While holding the SID B2 vertically, 0.75 mL of fresh sperm wash media was added to the SID central chamber. The SID unit was then gently lowered and nested in the C1 container, placing the mesh (porous barrier) in direct contact with the semen sample and a path for sperm to leave the native sample and enter the media in the SID chamber. The sample was then allowed to incubate at room temperature for one hour. At times 0, 5, 15, 30, and 60 minutes, a small (4 uL) aliquot of sample was taken from the middle of the media in the inner core, being careful not to allow the tip of the media to contact the SID mesh. The aliquot underwent an abbreviated semen analysis for concentration and motility, allowing the determination of the total motile sperm count inside the SID. In total 26 samples met the inclusion criteria and were processed for the study.

Statistical Analysis

Initial comparisons were made using a two-way analysis of variance comparing the two processing methods and the method over time. If differences were established, each combination of method and time was treated as an individual observation to allow a means of comparison for both method and time using either one-way analysis of variance or Student's t-tests as appropriate.

The holes in the porous barrier disclosed herein prevent the mass flow of media out of SID and into the reservoir of the sperm container reservoir and/or the sperm sample from the reservoir into the SID. The conventional swim up and other sperm purification methods (e.g., gradient wash and simple wash) operate by compacting the sperm at the bottom of a container by centrifugation and then adding media over top so the motile sperm swim out of the compacted mass of cells. The method disclosed herein eliminates the need for centrifugation, thus preventing damage to the sperm caused by the forces of centrifugation.

Studies done with done with porous barriers have 10 micron holes that demonstrated improved motility with human sperm by simple studies of sperm concentrations in the reservoir and the SID over time. Dye leakage studies demonstrated that the porous barriers with 30 micron holes may be too large to prevent mass flow between reservoir of the sample container and the chamber of the SID and were followed by simple studies comparing sperm concentrations between semen sample solutions and second solutions.

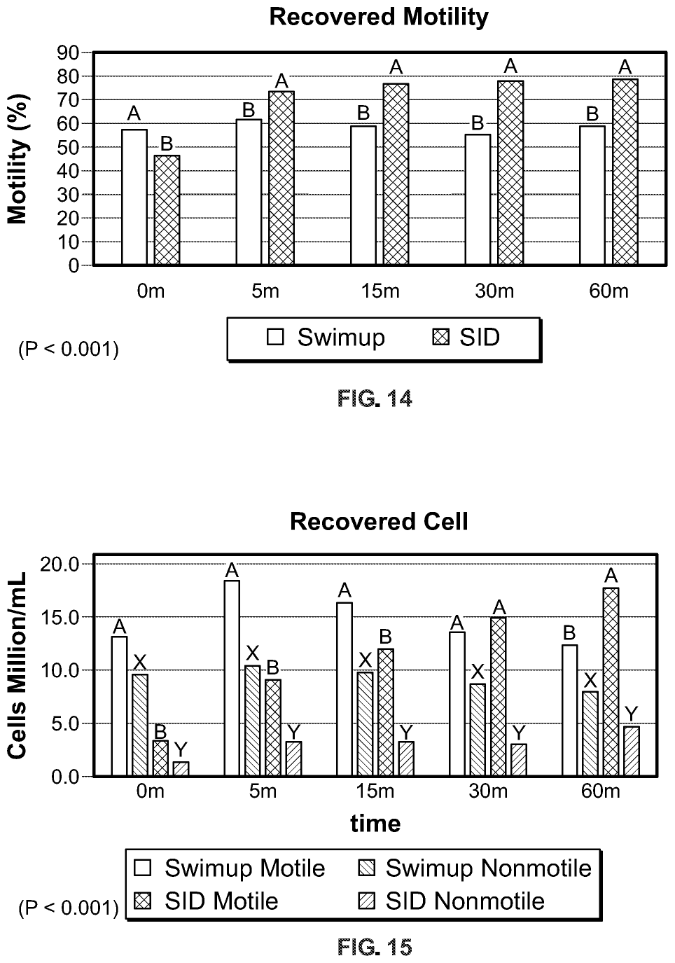

The motile concentration increased with time because of cells swimming into the SID (P<0.001; FIG. 14). The native samples in this study had motilities ranging from 29-88%. There is an obvious difference in recovered motility due to method (P<0.001) and time (P<0.001). There is also an interaction between time and treatment, with the SID trending upward over time while the swim-up prepared samples trended downward (P<0.001). While samples processed using the swim-up technique demonstrated an average motility of under 60% across the hour, once a significant number of cells had entered the SID (≥5 minutes), there was always a higher percentage of motile cells in the SID device.

FIG. 14 shows s comparison of the percentage of motile sperm cells seen over a one-hour time period in twenty-six paired samples split and processed using a swim-up procedure versus a new sperm isolation device using a barrier mesh between fluids (SID B2) one-step preparatory technique. Means with different superscripts within a time point are significantly different (P<0.001).

Further, the SID B2 produced a much “cleaner” sample compared to the swim-up, wherein cleaner means a lower content of nonmotile cells. The swim-up produced samples containing up to 40% nonmotile cells. Further, both the concentration of cells and the motility of cells peaked processed using swim-up peaked at 5 minutes and then decreased over the hour. In contrast, samples processed in the SID B2 had a linear increase in motile cells for at least the first hour after processing and demonstrated increased motility (P<0.001) as shown in FIG. 15.

FIG. 15 shows a comparison of the total number of motile and nonmotile sperm cells recovered over a one-hour time period in twenty-six paired samples, split and processed using a swim-up procedure versus a new sperm isolation device using a barrier mesh between fluids (SID B2) one-step preparatory technique demonstrating differences in recovery patterns between the two techniques (P<0.001). Means with different superscripts within a time point and cell type [motile (A,B) vs nonmotile (X,Y)] are significantly different (P<0.001).

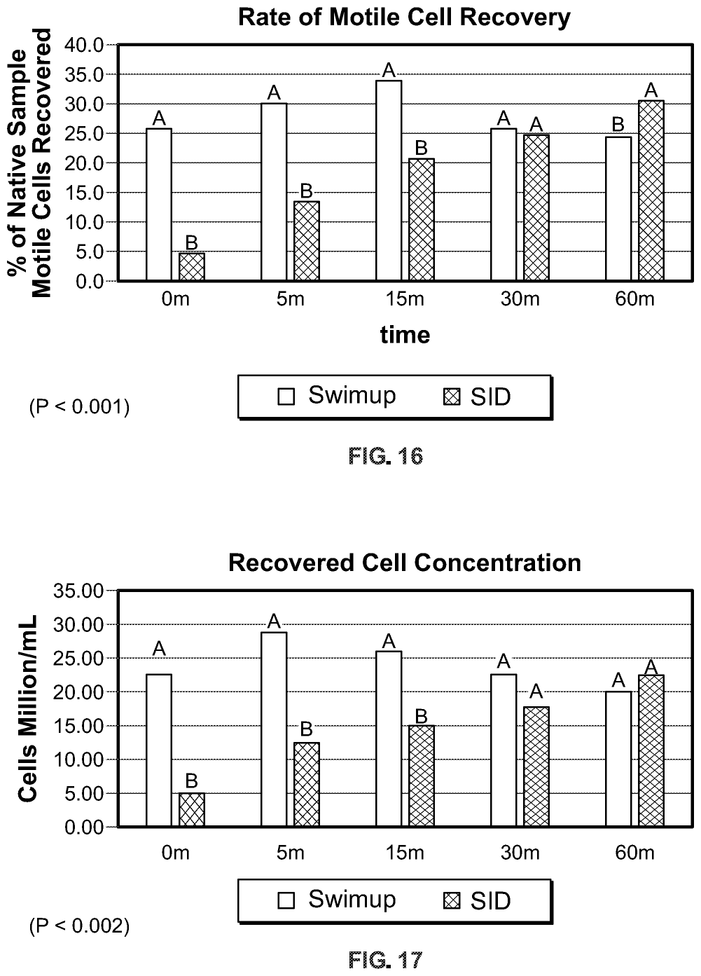

These outcomes were reflected in the percentage of recovered motile cells as a percentage of motile cells available from the native sample. While the highest number of motile cells in the swim-up occurred at 15 minutes, there was also the presence of 40% nonmotile cells in the sample as shown in FIG. 16. By contrast, the percentage of motile cells recovered with the SID B2 increased in a linear fashion and was over 80% motile, further, because of the barrier nature of the SID B2 device. The SID B2 eliminated all round cells, and preliminary data (not shown suggests a higher percentage of normal cells, suggesting the SID favors normal motile cells.

FIG. 16 shows a comparison of the percentage of motile sperm cells recovered from the available population in the native sample seen over a one-hour time period from twenty-six paired samples split and processed using a swim-up procedure versus a new sperm isolation device using a barrier mesh between fluids (SID B2) one-step preparatory technique demonstrated different patterns of sperm recovery (linear in the SID; P<0.001). Means with different superscripts within a time point are significantly different (P<0.001).

FIG. 17 shows a comparison of the change in number of total sperm cells (motile sand nonmotile) recovered from the available population in cell concentration of the native sample seen over a one-hour time in swim-up period from twenty-six paired samples split and processed using a swim-up procedure versus a new sperm isolation device using a barrier mesh between fluids (SID B2) one-step preparatory technique. Over time cell concentration demonstrated different patterns of sperm recovery (linear in the SID; P<0.001). The data demonstrates a linear increase in the sample prepared using the swim-up procedure increased rapidly at 0 and 5 minutes then progressively trailed off as cells fell out of solution. Where as the concentration of cells in the SID B2 device increased in a linear fashion for the entire hour.

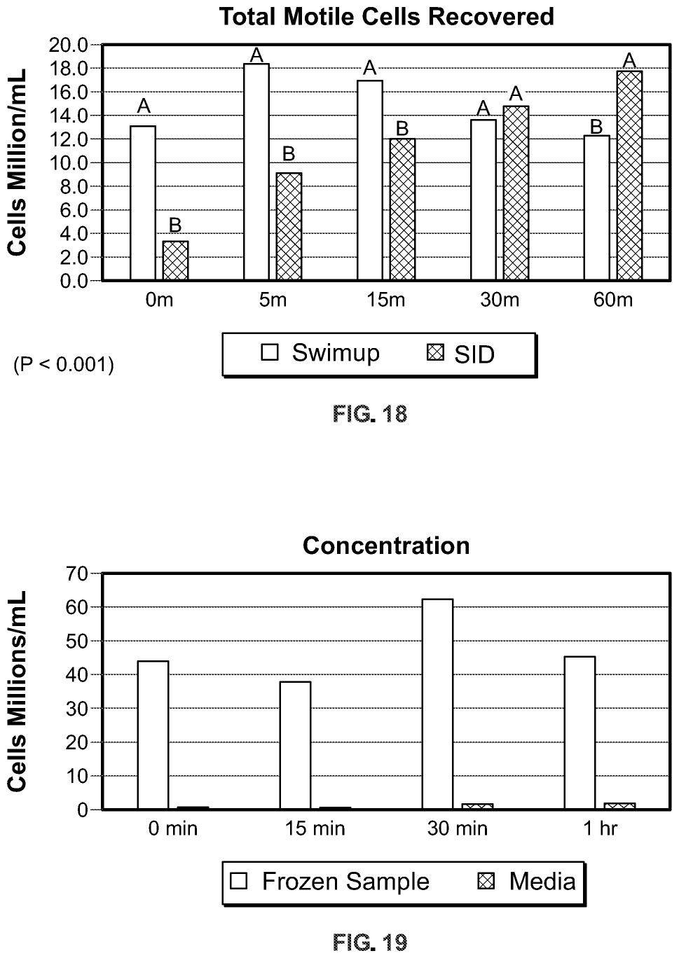

FIG. 18 shows a comparison of the change in number of total motile concentration sperm cells recovered from the available population in the native sample seen over a one-hour time in swim-up period from twenty-six paired samples split and processed using a swim-up procedure versus a new sperm isolation device using a barrier mesh between fluids (SID B2) one-step preparatory technique. Over time totile motile concentration demonstrated different patterns of sperm recovery (linear in the SID; P<0.001). The data demonstrates a linear increase in the sample prepared using the swim-up procedure increased at 0 and 5 minutes then progressively trailed off as cells lost motility presumably due to damage sustained during processing. Where as the total motile concentration of cells in the SID B2 device increased in a linear fashion for the entire hour yielding total motile concentrations sufficient over time (P<0.001). The data demonstrate adequate cell recover for ICSI, (5 min), convention IVF, (15 min) and IUI.

While a number of different techniques have developed for sperm processing, including commonly used simple wash, swim-up, density and centrifugation, and, more recently, barrier techniques, each presents challenges to obtaining a high-quality sample. The older techniques all rely on a centrifuged sample, which has been demonstrated to potentially disrupt cellular DNA through increasing reactive oxygen species. Further compounds used in density gradients may have issues with toxicity. Newer barrier techniques process only portions of the native sample, return only a small portion of the motile cells, and may require additional processing steps post-sample harvest. Data from the present study suggests that allowing cells to swim into the center well containing almost pure media preparation dramatically enhances the ratio of motile to nonmotile cells while confining larger round cells in the original collection vessel away from the motile population to be used in fertility treatment procedures with minimal technician effort.

Additionally, the new C1/B2 combined system described herein has many other potential advantages. The system is designed to work with an entire native ejaculate, thus increasing the total number of cells over methods that take only a portion of the sample for processing. It requires no centrifugation steps, and the original collection vessel limits oxygen exposure, lessening the chances of excess reactive oxygen species generation. Further, the collection container and SID materials contain embedded compounds known to scavenge excess reactive species, thus lowering the chances of the processing step causing DNA, membrane, or organelle damage during processing when compared to a standard specimen cup.

Frozen Samples

Methodology
1. Place 0.75 mL of fresh media in outer (collection vessel (in this case it has to be the protex or a similar shape container
2. Thaw and place 0.5 mL sample (total sample from cryovial) in SID. Note SID was modified to allow sampling of media in the collection vessel without disturbing SID
3. Nest SID in collection vessel
4. Incubate (room temp)
5. Harvest 4 uL from collection vessel and SID at times listed.

FIG. 19 shows data collected from a single frozen donor sample, where because of volume limitations, the sample was place in the SID and 0.75 mL of fresh media was place in vessel 1 (reverse of protocol with fresh sample). Sample from the SID and the vessel were collected at times 0, 15, 30 and 60 minutes (top of SID was modified to allow access to media without disturbing SID. Measurable concentrations of sperm cells were detect in the fresh media (in vessel) at all time points past 0 minutes. and increase in a linear fashion over time.

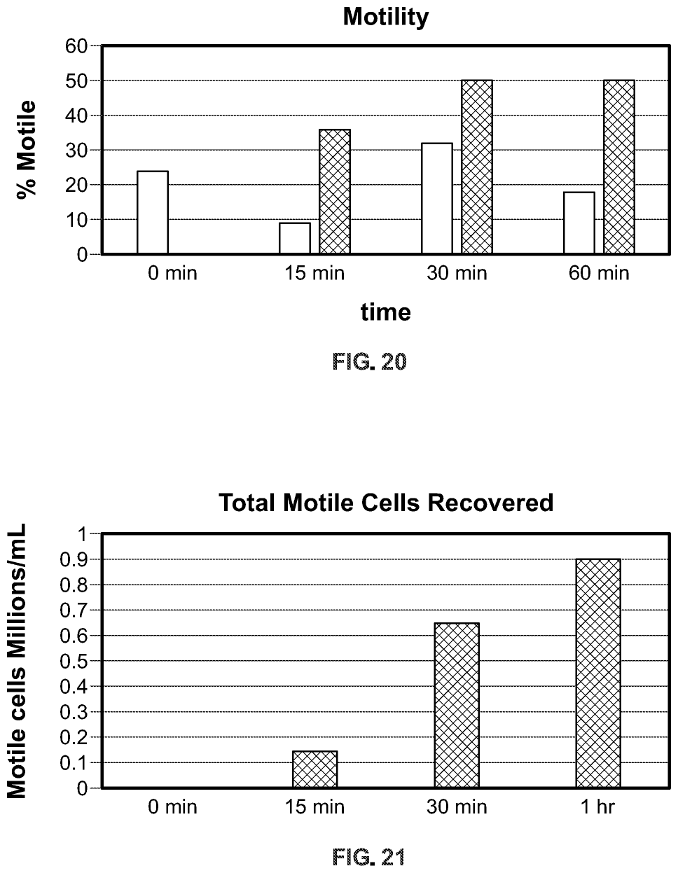

FIG. 20 shows data collected from a single frozen donor sample, where because of volume limitations, the sample was place in the SID and 0.75 mL of fresh media was place in vessel 1 (reverse of protocol with fresh sample). Sample from the SID and the vessel were collected at times 0, 15, 30 and 60 minutes (top of SID was modified to allow access to media without disturbing SID. As expected, there was no motility at time 0 in the SID as there were no cells. However, the motility seen in the fresh media was higher (18-32% higher) than that seen in the original sample inside the SID at all time points past 0 mins.

FIG. 21 shows data collected from a single frozen donor sample, where because of volume limitations, the sample was place in the SID and 0.75 mL of fresh media was place in vessel 1 (reverse of protocol with fresh sample). The graph represented the total number of motile cells recovered in the fresh media demonstrating a sample usable for either conventional IVF or ISCI.

For the sake of brevity, only certain ranges are explicitly disclosed herein. However, in addition to recited ranges, any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

Although the present invention and its advantages have been described in detail, it should be understood that various changes, substitutions, and alterations can be made herein without departing from the spirit and scope of the invention as defined by the appended claims. All patents, test procedures, and other documents cited in this application are fully incorporated herein by reference for all jurisdictions in which such incorporation is permitted. Moreover, the scope of the present application is not intended to be limited to the particular embodiments of the processes, equipment, means, methods, and/or steps described in the specification. As one of the ordinary skill in the art will readily appreciate from the disclosure of the present invention, processes, equipment, means, methods, and/or steps, presently existing or later to be developed that perform substantially the same function or achieve substantially the same result as the corresponding embodiments described herein, may be utilized according to the present invention. It is possible within the embodiments to have a SID that contains multiple porous barriers of the same or different hole sizes or having two or more SID's used in combination with one or more vessels. It is also within the embodiments of the invention to have an array of two or more connected apparatus of the invention for conducting the method on multiple semen samples from the same or different species. Accordingly, the appended claims are intended to include within their scope such processes, equipment, means, methods, and/or steps.

What is claimed is:

1. An apparatus for passively isolating a population of motile sperm from a semen sample solution, the apparatus comprising:
a) a first vessel for receiving the semen sample solution, and
b) a sperm isolation device (SID) for receiving a nutrient media, the device comprising a frame and a porous barrier, the porous barrier comprising a plurality of holes, wherein each hole has a passthrough dimension in a range of from 6 microns to 30 microns.

2. The apparatus of claim 1, wherein each hole has a hydraulic diameter in the range of from 6 microns to 30 microns.

3. The apparatus of claim 1, wherein
a) the first vessel has a reservoir suitable for receiving the semen sample solution;
b) the SID has a chamber suitable for receiving the nutrient media;
c) the SID fits within the first vessel such that the reservoir and the chamber are fluidly connected through at least a portion of the holes in the porous barrier when:
i) the first vessel has received the semen sample solution;
ii) the SID has received the nutrient media; and
iii) the SID is engaged with the first vessel.

4. The apparatus of claim 1, wherein the SID is substantially conical and adapted for limiting and stabilizing its insertion within the first vessel.

5. The apparatus of claim 1, wherein:
a) the frame of the SID has a rim member attached to a solid base by at least two downwardly extending supports; and
b) the porous barrier is positioned between each pair of adjacent supports and extends from the solid base to the rim member.

6. The apparatus of claim 1, wherein the apparatus does not include any means for promoting fluid flow between the reservoir in the first vessel and the chamber in the SID.

7. The apparatus of claim 1, wherein the apparatus comprises more than one SID disposed within the first vessel.

8. A method for passively isolating a population of motile sperm from a semen sample solution, the method comprising:
(i) introducing the semen sample solution into a first vessel;
(ii) introducing a harvest media into a sperm isolation device (SID) comprising a porous barrier; and
(iii) inserting the SID in the semen sample solution to submerge at least a portion of the porous barrier;
wherein:
the porous barrier comprises a plurality of holes, each having a passthrough dimension in a range of from 6 microns to 30 microns; and
a population of motile sperm in the semen sample solution migrate through the holes in the porous barrier into the harvest media.

9. The method of claim 8, wherein each hole has a hydraulic diameter in the range of from 6 microns to 30 microns.

10. The method of claim 8, wherein the semen sample solution further comprises a sample media, and the sample media and the harvest media are the same or different.

11. The method of claim 8, wherein step (iii) is performed in a manner to prevent or minimize transfer through the porous barrier of the semen sample solution into the SID and/or the harvest media into the semen sample solution.

12. The method of claim 8, wherein in step (ii) the harvest media is added to the SID via a transfer device, wherein the transfer device does not contact the porous barrier.

13. The method of claim 8, wherein migration of the motile sperm through the porous barrier into the harvest media is a result of chemotaxis.

14. The method of claim 8, wherein:
a) after step (ii) and before step (iii), the semen sample solution has a first concentration of sperm, and the harvest media has no concentration of sperm;
b) at a threshold time period after step (iii), the semen sample solution has a second concentration of sperm, and the harvest media has a third concentration of sperm; and
c) the second concentration is less than the first concentration.

15. The method of claim 14, wherein the threshold time period is in the range of from 5 minutes to 3 hours.

16. The method of claim 8, wherein:
a) after step (ii) and before step (iii), the semen sample solution comprises a first population of sperm having a first motility, and the harvest media comprises no sperm;
b) at a threshold time period after step (iii), the semen sample solution comprises a second population of sperm having a second motility, and the harvest media has a third population of sperm having a third motility;
c) the second population of sperm is less than the first population of sperm;
d) the third population of sperm is the difference between the second population of sperm and the first population of sperm; and
e) the third motility is greater than the first or second motility.

17. The method of claim 16, wherein the threshold time period is in the range of from 5 minutes to 3 hours.

18. The method of claim 8, further comprising:
(iv) withdrawing a portion of the harvest media comprising motile sperm at a threshold time period after step (iii).

19. The method of claim 18, wherein the threshold time period is in the range of from 5 minutes to 3 hours.

20. The method of claim 18, wherein the threshold time period is in the range of from 10 and 20 minutes for direct use in in vitro fertilization, in vitro fertilization with intracytoplasmic sperm injection (ICSI), intrauterine insemination (IUI), or other means of artificial insemination without centrifugation.

21. The method of claim 18, wherein the method is conducted:
a) at a temperature in the range of from 5° C. to 40° C.;
b) with no movement of the apparatus in step (iv);
c) with no agitation of the semen solution in the first vessel and/or the harvest media in the SID; or
d) a combination thereof.

22. The method of claim 8, wherein each hole has a passthrough dimension in the range of from 6 microns to 12 microns, and the semen sample solution comprises a semen sample from a human.

23. The method of claim 8, wherein each hole has a passthrough dimension in the range of from 7 microns to 30 microns, and the semen sample solution comprises a semen sample from an animal.

24. The method of claim 8, wherein the semen sample solution comprises a volume ratio of the sample media to the semen sample in the range of from 1:5 to 1:1.

25. The method of claim 8, wherein the volume ratio of the semen sample solution to the fresh media is in the range of from 2:3 to 10:1.

26. The method of claim 8, wherein step (iii) is performed prior to step (ii).

27. The method of claim 8, wherein the semen sample has not been agitated, centrifuged, or otherwise treated prior to step (i).

28. A method for passively isolating a population of motile sperm from a semen sample solution, the method comprising:
(i) introducing a harvest media into a first vessel;
(ii) introducing the semen sample solution into a sperm isolation device (SID) comprising a porous barrier; and
(iii) inserting the SID in the harvest media to submerge at least a portion of the porous barrier;
wherein:
the porous barrier comprises a plurality of holes, each having a passthrough dimension in a range of from 6 microns to 30 microns; and
a population of motile sperm in the semen sample solution migrate through the holes in the porous barrier into the harvest media.

29. The method of claim 28, wherein:
a) after step (ii) and before step (iii), the semen sample solution comprises a first population of motile sperm, and the harvest media comprises no sperm;
b) at a threshold time period after step (iii), the semen sample solution comprises a second population of motile sperm, and the harvest media has a third concentration of motile sperm; and
c) the second population of motile sperm is in the range of from 5% to 30% of the first population of motile sperm.

30. The method of claim 29, wherein the threshold time period is in the range of from 5 minutes to 3 hours.

31. The method of claim 28, wherein the semen sample was frozen, is less than 1 mL, or a combination thereof.

32. A sperm isolation device (SID) for passively isolating a population of motile sperm from a semen sample solution, the device comprising:
a) a frame; and
b) a porous barrier comprising a plurality of holes, each having a passthrough dimension in a range of from 6 microns to 30 microns.

33. The sperm isolation device of claim 32, wherein:
a) the frame and the porous barrier form a chamber having an opening at the upper end suitable for receiving a portion of a media;
b) the SID has a lower portion suitable for insertion into a semen sample solution; and
c) the chamber and the solution are fluidly connected through at least a portion of the holes in the in the porous barrier when:
i) the sperm isolation device has received the portion of media; and
ii) the SID is inserted into the solution.

34. The sperm isolation device of claim 32, wherein the frame of the SID is adapted for limiting and/or stabilizing the vertical position of the sperm isolation device when inserted into a semen sample solution.

35. The sperm isolation device of claim 32, wherein:
a) the frame of the sperm isolation device has a rim member attached to a solid base by at least two downwardly extending supports; and
b) the porous barrier is positioned between each pair of adjacent supports and extends from the solid base to the rim member.

36. A method for using the sperm isolation device of claim 32, the method comprising:
(i) introducing a harvest media into the chamber of the SID;
(ii) inserting the lower portion of the SID into a semen sample solution comprising a first amount of motile sperm; and
(iii) after a threshold time period, withdrawing a portion of the harvest media from the chamber with a transfer device, wherein the harvest media comprises a second amount of motile sperm.

37. The method of claim 36, wherein the threshold time period is in the range of from 5 minutes to 3 hours.

38. The method of claim 36, wherein the threshold time period is in the range of from 10 and 20 minutes for direct use in testing. in vitro fertilization (IVF), in vitro fertilization with intracytoplasmic sperm injection (IC SI), intrauterine insemination (IUI), or other means of artificial insemination without centrifugation.

39. The method of claim 36, further comprising introducing the enhanced sperm sample to an egg for fertilization.

40. The method of claim 36, wherein each hole has a passthrough dimension in a range of from 6 microns to 12 microns, and the semen sample solution comprises a semen sample from a human.

41. The method of claim 36, wherein each hole has a passthrough dimension in a range of from 7 microns to 30 microns, and the semen sample solution comprises a semen sample from an animal.
